(19) United States
(12) Patent Application Publication
(10) Pub. No.: US 2025/0122494 A1
(43) Pub. Date: Apr. 17, 2025

(54) METHODS FOR SEPARATING MOLECULAR SPECIES OF GUANINE-RICH OLIGONUCLEOTIDES

(71) Applicant: AMGEN INC., Thousand Oaks, CA (US)

(72) Inventors: Robert J. DUFF, Moorpark, CA (US); Helena SCHILLINGER, Thousand Oaks, CA (US); Jennifer LIPPENS, Newbury Park, CA (US)

(73) Assignee: AMGEN INC., Thousand Oaks, CA (US)

(21) Appl. No.: 18/695,949

(22) PCT Filed: Sep. 29, 2022
(86) PCT No.: PCT/US2022/045152

§ 371 Date: Mar. 27, 2024

Related U.S. Application Data
(60) Provisional application No. 63/250,650, filed on Sep. 30, 2021.

(51) Int. Cl. C12N 15/10

(52) U.S. Cl. CPC C12N 15/101

(57) ABSTRACT

Provided herein are methods of separating molecular species of a guanine-rich oligonucleotide from a mixture of molecular species, wherein at least one molecular species of the mixture is a quadruplex formed from the guanine-rich oligonucleotide. In exemplary embodiments, the methods comprise (a) applying the mixture to a chromatographic matrix comprising a hydrophobic ligand, wherein said hydrophobic ligand comprises C4 to C8 alkyl chains, wherein molecular species bind to the hydrophobic ligand and (b) applying a mobile phase which comprises a gradient of acetate and a gradient of acetonitrile but no cationic ion pairing agent to the chromatographic matrix to elute molecular species of the guanine-rich oligonucleotide. In exemplary aspects, the guamne-rich oligonucleotide elutes in a first set of elution fractions and a quadruplex formed from the guanine-rich oligonucleotide elutes in a second set of elution fractions.

### CROSS REFERENCE TO RELATED APPLICATIONS

The benefit under 35 U.S.C. § 119(e) of U.S. Provisional Patent Application No. 63/250,650, filed on Sep. 30, 2021, is hereby claimed, and the disclosure thereof is hereby incorporated by reference herein

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 8 KB XML file named “A-2735-WO01-SEC_Sequence_Listing.XML”; created on Sep. 9, 2022.

### FIELD OF THE INVENTION

The present invention relates to the fields of nucleic acid purification and analytical detection and characterization. In particular, the invention relates to methods for separating molecular species of a guanine-rich oligonucleotide from a mixture of molecular species, wherein at least one molecular species of the mixture is a quadruplex formed from the guanine-rich oligonucleotide. The methods allow for the separation, detection, and purification of each individual molecular species of the guanine-rich oligonucleotide in the mixture, including single strand oligonucleotides as well as higher-order structures, such as duplexes and quadruplexes.

### BACKGROUND

Treatment of various cell types with guanine-rich (G-rich) oligonucleotides has been reported to lead to a diverse array of biological effects, including inhibition of cell proliferation, induction of cell death, changes in cellular adhesion, inhibition of protein aggregation, and antiviral activity, (Bates et al., Exp Mol Pathol 86(3): 151-164 (2009)). Recently, multiple synthetic G-rich oligonucleotides have been investigated as therapeutic agents for various human diseases.

G-rich oligonucleotides can associate intermolecularly or intramolecularly to form four-stranded or quadruple-stranded (G4) or “quadruplex” structures. These structures form via the formation of G-quartets, in which four guanines establish a cyclic pattern of hydrogen bonds. Structurally, the tetrameric aggregate consists of planar assemblies allowing both anti or syn glycosidic conformation; tetrad guanines from G-strands with the same direction, i.e., parallel strands, adopt the same glycosidic conformation, whereas those from G-strands with the opposite direction, i.e., antiparallel strands, adopt different glycosidic conformations. The orientation of the base (anti or syn) may contribute to stability (Huppert et al., Chemical Society Reviews, 37(7), pp. 1375-1384 (2008); Burge et al., Nucleic Acids Research, 34(19), pp. 5402-5415 (2006); and Lane, Biochimie, 94(2), pp. 277-286 (2012)).

As a result of the orientation of the guanines residues, G-rich DNA quadruplex structures are intrinsically very unstable. The instability of these structures is, at first, counterintuitive, despite the well-known observation that quadruplexes require univalent ions of the correct size to fold. Cations, particularly K⁺ and to a lesser extent Na⁺, and even NH₄⁺ stabilize stacked G-tetrads by coordinating with tetrad-guanine O6 atoms. However, melting profiles reveal nothing about quadruplex topology and structure, though parallel topologies are usually more stable than antiparallel ones, and potassium ions produces more stable complexes than sodium (Sannohe and Sugiyama, Current protocols in nucleic acid chemistry, 40(1), pp. 17-2 (2010); and Rachwal and Fox, Methods, 43(4), pp. 291-301 (2007)).

The stability of the G-quadruplex is governed by various parameters, such as electrostatics, base stacking, hydrophobic interactions, hydrogen bonding, and van der Waals forces. Thermal stability increases as the dielectric constant of the solvent decreases (Smimov and Shafer, Biopolymers: Original Research on Biomolecules, 85(1), pp. 91-101 (2007). Thermodynamic assessment for this equilibrium is based on melting profiles of the higher order structure, in which the denaturation process of the G4 structure is monitored by typically spectroscopic methods (Yang, D. and Lin, C. eds., 2019. G-quadruplex Nucleic Acids: Methods and Protocols. Humana Press). Quadruplexes may also be studied by x-ray, NMR, CD, and UV techniques. Discernment of strand orientation can be assessed through the absorbance at 295 nm (Mergny et al., FEBS Lett. 435, 74-78 (1998); Mergny and Lacroix, Oligonucleotides. 2003; 13(6):515-537; Mergny and Lacroix, Current protocols in nucleic acid chemistry, 37(1), pp. 17-1 (2009); Majhi et al., Biopolymers: Original Research on Biomolecules, 89(4), pp. 302-309 (2008); Petraccone et al., Current Medicinal Chemistry-Anti-Cancer Agents, 5(5), pp. 463-475 (2005); Darby et al., Nucleic Acids Research, 30(9), pp. e39-e39 (2002)).

The quadruplex structures of G-rich oligonucleotides are associated with unusual biophysical and biological properties. Increasing evidence shows that quadruplex structures are present in vivo, and it has been suggested that these structures play a role in various physiological functions, such as in DNA replication, telomere maintenance, and gene expression. Rhodes and Lipps, Nucleic Acids Research, Vol. 43: 8627-8637, 2015.

To gain a better understanding of these structures and to ultimately harness the therapeutic potential of G-rich oligonucleotides that form quadruplex structures, researchers need to be able to detect, characterize, isolate, and purify these molecules. Generally, most approaches for purifying guanine-rich oligonucleotides or separating them from associated impurities aim to disrupt secondary interactions, such as quadruplex formation, by using elevated temperatures, high pH buffers, or introducing chaotropic agents or organic modifiers. Such strongly denaturing conditions promote single-strand formation. The single strand could then be purified, but the quadruplex would then need to be assembled from the purified single strands. From an analytical perspective, strong denaturing conditions may affect accurate quantitation of the quadruplex structure or other impurities of higher order structure present in an analytical sample.

In view of the foregoing, there remains a need for efficient methods of purifying or separating guanine-rich oligonucleotides from the quadruplexes formed therefrom and other impurities.

### SUMMARY OF THE INVENTION

The present invention relates to a separation method for guanine-rich oligonucleotides that tend to form quadruplex structures. The invention is based, in part, on the discovery that the formation of quadruplex structures from the guanine-rich oligonucleotides can be chromatographically separated by the presently disclosed methods which employ a chromatographic matrix comprising a hydrophobic ligand comprising C4 to C8 alkyl chains and a mobile phase comprising a gradient of acetate and a gradient of acetonitrile. Such methods, as demonstrated herein, allow high resolution separation between not only the guanine-rich oligonucleotide and the quadruplex, but between other predominant molecular species of the guanine-rich oligonucleotide. Advantageously, the methods of the present disclosure may be used to achieve high resolution separation for the guanine-rich oligonucleotide, its complementary strand, the quadruplex and the duplex comprising the guanine-rich oligonucleotide and its complementary strand.

The present inventors have surprisingly found that by excluding a cationic ion pairing agent, such as triethylamine (TEA), from the mobile phase, high resolution among the peaks corresponding to the molecular species of the guanine-rich oligonucleotide may be achieved using a reverse-phase (i.e. hydrophobic) stationary phase.

Accordingly, the present invention provides methods of separating molecular species of a guanine-rich oligonucleotide from a mixture of molecular species. In exemplary embodiments, at least one molecular species of the mixture is a quadruplex formed from the guanine-rich oligonucleotide. In exemplary embodiments, the method comprises (a) applying the mixture to a chromatographic matrix comprising a hydrophobic ligand, wherein said hydrophobic ligand comprises C4 to C8 alkyl chains, wherein molecular species bind to the hydrophobic ligand; and (b) applying a mobile phase which comprises a gradient of acetate and a gradient of acetonitrile to the chromatographic matrix to elute molecular species of the guanine-rich oligonucleotide. In exemplary aspects, each molecular species elutes at a time distinct from the time at which a different molecular species elutes. For example, in exemplary instances, the guanine-rich oligonucleotide elutes at a distinct time at which the quadruplex elutes. In various aspects, the guanine-rich oligonucleotide elutes in a first set of elution fractions and the quadruplex elutes in a second set of elution fractions.

In exemplary aspects, the guanine-rich oligonucleotide is a sense strand or an antisense strand of a small interfering RNA (siRNA). In exemplary instances, the mixture comprises single-stranded molecular species and/or double-stranded molecular species. Optionally, the mixture comprises one or more molecular species selected from the group consisting of: an antisense single strand, a sense single strand, a duplex, and a quadruplex. In various aspects, the guanine-rich oligonucleotide is the antisense single strand. In various instances, the duplex comprises the antisense single strand and the sense single strand. In exemplary aspects, the mixture comprises all of the following molecular species: an antisense single strand, a sense single strand, a duplex, and a quadruplex. Optionally, each molecular species elutes in a fraction separate from that of another molecular species. In exemplary aspects, the duplex elutes in a first set of elution fractions, the sense strand elutes in a second set of elution fractions, the antisense strand elutes in third set of elution fractions, and the quadruplex elutes in a fourth set of elution fractions. In various aspects, the resolution of the separation of the peaks of each molecular species (e.g., the resolution of the separation between the peak of the duplex and the peak of the sense single strand) is at least or about 1.0, optionally, at least or about 1.1, at least or about 1.2, at least or about 1.3, or at least or about 1.4. In various aspects, the resolution of the separation of the peaks of each molecular species is at least or about 1.5, optionally, at least or about 1.6, at least or about 1.7, at least or about 1.8, or at least or about 1.9. Optionally, the resolution of the separation of the peaks corresponding to each molecular species (e.g., the resolution of the separation between the peak of the duplex and the peak of the sense single strand) is at least or about 2.0 (e.g., at least or about 2.1, at least or about 2.2, at least or about 2.3, at least or about 2.4. In various aspects, the resolution of the separation is at least or about 2.4. In exemplary instances, the resolution is at least or about 2.5, at least or about 3.0, or at least or about 4.0. Optionally, the resolution of the separation between the peak of the duplex and the peak of the sense strand is at least 4.0. In various aspects, the limit of quantitation (LOQ) of each molecular species is about 0.03 mg/mL to about 0.08 mg/mL, when the signal-to-noise ratio is greater than or equal to 10.0. In various instance, the LOQ is about 0.08 mg/ml when the signal-to-noise ratio is greater than or equal to 10.0.

In various instances, the mixture is prepared in a solution comprising one or more of: water, a source of acetate, a source of potassium, and sodium chloride. The source of acetate is ammonium acetate, sodium acetate, potassium acetate in certain aspects. Optionally, the source of potassium is potassium phosphate. In various aspects, the solution comprises about 50 mM to about 150 mM acetate or potassium. In various instances, the solution comprises about 75 mM to about 100 mM of ammonium acetate, sodium acetate, or potassium acetate. In exemplary aspects, the solution comprises potassium phosphate and sodium chloride.

In certain embodiments, the chromatographic matrix comprises a hydrophobic ligand comprising C4 alkyl chains, C6 alkyl chains, or C8 alkyl chains. Optionally, the hydrophobic ligand comprises C4 alkyl chains. In exemplary aspects, the chromatographic matrix is housed in a chromatographic column having an internal diameter of 2.1 mm and/or a column length of about 50 mm. In exemplary instances, the column temperature is about 20° C. to about 35° C., optionally, about 30° C. In various instances, the chromatographic matrix comprises ethylene bridged hybrid (BEH) particles. Optionally, the BEH particles have a particle diameter of about 1.7 μm or about 3.5 μm

In some embodiments, the gradient of acetate in the mobile phase is made with an acetate stock solution comprising about 50 mM to about 150 mM acetate. Optionally, the acetate stock solution comprises about 70 mM to about 80 mM acetate, optionally, about 75 mM acetate. In various aspects, the acetate stock solution comprises about 90 mM to about 110 mM acetate, optionally, about 100 mM acetate. In various instances, the acetate is ammonium acetate, sodium acetate or potassium acetate. In exemplary aspects, the pH of the acetate stock solution is about 6.5 to about 7.0, optionally, about 6.7, about 6.8, about 6.9, or about 7.0. In exemplary aspects of the present disclosure, the mobile phase comprises a decreasing gradient of the acetate and an increasing gradient of acetonitrile. In exemplary instances, the gradient of acetate starts with a maximum concentration and gradually decreases to a minimum concentration over a first time period. Optionally, the first time period is about 18 to about 19 minutes, and, alternatively, the first time period is about 22 minutes to about 26 minutes. In various aspects, after the first time period, the mobile phase increases to the maximum concentration of acetate, optionally, about 0.1 to about 3 minutes after the gradient reaches the minimum concentration of acetate. In various aspects, the gradient of acetonitrile starts with a minimum concentration and gradually increases to a maximum concentration over the first time period. Optionally, after the first time period, the mobile phase decreases to the minimum concentration of acetonitrile. Optionally, the mobile phase decreases to the minimum concentration of acetonitrile about 0.1 to about 3 minutes after the gradient of acetonitrile reaches the maximum concentration of acetonitrile. In certain aspects, the method of the present disclosure comprises applying the mobile phase to the chromatographic matrix according to the following conditions:

| Time (min) | Acetate (%) | Acetonitrile |
|---|---|---|
| 0 | 93 | 7 |
| 5 | 88 | 12 |
| 8 | 88 | 12 |
| 11 | 86 | 14 |
| 18 | 70 | 30 |
| 19 | 70 | 30 |
| 21 | 93 | 7 |
| 26 | 93 | 7 |

In alternative or additional aspects, the method comprises applying the mobile phase to the chromatographic matrix according to the following conditions:

| Time (min) | Acetate (%) | Acetonitrile (%) |
|---|---|---|
| 0 | 92 | 8 |
| 2 | 90 | 10 |
| 18 | 86 | 14 |
| 26 | 70 | 30 |
| 26.1 | 92 | 8 |
| 30 | 92 | 8 |

In alternative or additional aspects, the method comprises applying the mobile phase to the chromatographic matrix according to the following conditions:

| Time (min) | Acetate (%) | Acetonitrile (%) |
|---|---|---|
| 0.0 | 92 | 8 |
| 2.0 | 90 | 10 |
| 18.0 | 86 | 14 |
| 22.0 | 78 | 22 |
| 22.1 | 20 | 80 |
| 24.0 | 20 | 80 |
| 24.1 | 92 | 8 |
| 30.0 | 92 | 8 |

In exemplary aspects, the mobile phase does not comprise a cationic ion pairing agent, e.g., TEA. The total run time is, in various instances, at least about 25 minutes and less than 40 minutes, optionally, less than 35 minutes, optionally, less than or equal to 30 minutes. In various instances, the run time is about 22 minutes to about 26 minutes. In various aspects, the flow rate of the mobile phase is about 0.5 ml/min to about 1.0 m/min, optionally, about 0.7 ml/min to about 0.8 ml/min.

In exemplary aspects, the guanine-rich oligonucleotide comprises about 19 to about 23 nucleotides. In exemplary instances, the guanine-rich oligonucleotide and one or more of the molecular species thereof in the mixture comprises one or more modified nucleotides. Optionally, the one or more modified nucleotides are 2′-modified nucleotides, such as 2′-O-methyl modified nucleotides, 2′-fluoro modified nucleotides, deoxynucleotides, or combinations thereof. In various aspects, the guanine-rich oligonucleotide and one or more of the molecular species thereof in the mixture comprises synthetic intemucleotide linkages, such as phosphorothioate linkages.

The present invention also provides a method of determining the purity of a sample comprising a guanine-rich oligonucleotide drug substance or drug product. In exemplary embodiments, the method comprises separating molecular species of the guanine-rich oligonucleotide in accordance with the presently disclosed methods of separating molecular species of the guanine-rich oligonucleotide. In various aspects, the sample is an in-process sample and the method is used as part of an in-process control assay or as an assay for ensuring the manufacture of the G-rich oligonucleotide is being carried out without substantial impurities. In various instances, the sample is a lot sample and the method is used as part of a lot release assay. In various aspects, the sample is a stressed sample or a sample that has been exposed to one or more stresses, and the method is a stability assay. Accordingly, the present invention provides a method of testing stability of a guanine-rich oligonucleotide drug substance or drug product, comprising applying stress to a sample comprising the guanine-rich oligonucleotide drug substance or drug product and determining the purity of the sample according to a method of the present disclosure. In exemplary instances, the presence of impurities in the sample after the one or more stresses indicates instability of the G-rich oligonucleotide under the one or more stresses.

### BRIEF DESCRIPTION OF THE DRAWINGS

FIG. 1 depicts the structure of olpasiran schematically. The top strand listed in the 5′ to 3′ direction is the sense strand (SEQ ID NO: 3) and the bottom strand listed in the 3′ to 5′ direction is the antisense strand (SEQ ID NO: 4). Black circles represent nucleotides with a 2′-O-methyl modification, white circles represent nucleotides with a 2′-deoxy-2′-fluoro (“2′-fluoro”) modification, and the gray circle represents a deoxyadenosine nucleotide linked to the adjacent nucleotide via a 3′-3′ linkage (i.e. inverted). Gray lines connecting the circles represent phosphodiester linkages, whereas the black lines connecting the circles represent phosphorothioate linkages. A trivalent GaINAc moiety having the depicted structure is represented by R1 and is covalently attached to the 5′ end of the sense strand by a phosphorothioate linkage.

FIG. 2A is an exemplary chromatogram of the peaks for the antisense, sense and duplex molecular species separated using a chromatographic matrix comprising a C18 hydrophobic ligand and a mobile phase comprising HAA/acetonitrile/methanol (MP A) and HAA/acetonitrile (MP B), as described in Study 1 of Example 1.

FIG. 2B is a series of chromatograms obtained from eluting olpasiran samples from a Waters XBridge BEH C4 column wherein the mobile phase MP A was 95 mM HFIP/8 mM TEA/24 mM tert-butylamine and MP B was acetonitrile, as described in Study 2 of Example 1.

Each of FIGS. 2C-2G is a series of exemplary chromatograms obtained from eluting samples of olpasiran from a Waters XBridge BEH C4 column wherein the mobile phase comprised different alkylamines and/or different concentrations of TEA or HFIP, as described in Table 3 of Study 3A.

Each of FIGS. 2H-21 is a series of exemplary chromatograms obtained from eluting samples of olpasiran from a Waters XBridge BEH C4 column wherein the mobile phase components and/or the mobile gradient conditions were modified, as described in Studies 3D and 3E.

FIGS. 2J and 2K provide exemplary chromatograms at each of the tested column temperatures. FIG. 2J shows the peaks for the sense and duplex, while FIG. 2K shows the peaks for the antisense and quadruplex.

FIG. 2L is a series of chromatograms obtained from eluting samples of olpasiran from a column having a longer column length (100 mm). FIG. 2M is a series of chromatograms obtained from eluting samples of olpasiran from a column having a shorter column length (50 mm).

FIG. 3 is a graph of the % peak area for the duplex peak plotted as a function of the duplex concentration.

FIG. 4 is a series of chromatograms showing the peaks of the antisense strand and quadruplex when the olpasiran sample is prepared in water (A10A-W), ammonium acetate (A10A-N), or HFIP/TEA (A10A-H).

FIG. 5 is a pair of chromatograms showing the peaks for the antisense strand and quadruplex when the olpasiran sample is prepared in water and heated (bottom) or not heated (top).

FIG. 6 is a pair of chromatograms showing the peaks for the antisense strand and quadruplex when the olpasiran sample is prepared in ammonium acetate and heated or not heated.

FIG. 7 is an exemplary chromatogram of the antisense/quadruplex equilibrium in heated samples comprising a water solvent.

FIG. 8 is a graph of the % peak area for the quadruplex peak plotted as a function of the concentration.

FIG. 9A and FIG. 9B are overlay and stacked chromatograms obtained when carrying out an exemplary method of the present disclosure according to a first method described in Example 6.

FIG. 10A and FIG. 10B are overlay and stacked chromatograms obtained when carrying out an exemplary method of the present disclosure according to a second method described in Example 6.

FIG. 10C and FIG. 10D are overlay and stacked chromatograms obtained when carrying out an exemplary method of the present disclosure according to a third method described in Example 6.

Each of FIGS. 11-14 is a graph of the % peak area plotted as a function of concentration for the duplex, sense strand, antisense strand and quadruplex, respectively.

FIG. 15 is a scheme of the study carried out to test the effect of heating-cooling treatment.

FIGS. 16A and 16B show the overlay chromatograms of the antisense strand solutions prepared in water before and after the heating-cooling treatment. FIGS. 17A and 17B show the overlay chromatograms of the antisense strand solutions in 75 mM ammonium acetate buffer before and after the heating-cooling treatment.

FIG. 18 is an MS spectrum associated with the proposed antisense single strand provide a narrow charge state distribution of the 3+ and 4+ charge states.

FIG. 19 an MS spectrum was pulled from the concentrated G-quadruplex sample, MS signals were observed at higher m/z.

FIG. 20 is a graph of the intensity plotted as a function of size as measured by dynamic light scattering (DLS).

FIG. 21 is a graph of the volume plotted as a function of size as measured by DLS.

### DETAILED DESCRIPTION

The present invention provides methods for separating a guanine-rich oligonucleotide from a mixture of molecular species. In exemplary aspects, at least one molecular species of the mixture is a quadruplex formed from the guanine-rich oligonucleotide. In exemplary embodiments, the method comprises (a) applying the mixture to a chromatographic matrix comprising a hydrophobic ligand, wherein said hydrophobic ligand comprises C4 to C8 alkyl chains, wherein molecular species bind to the hydrophobic ligand; and (b) applying a mobile phase which comprises a gradient of acetate and a gradient of acetonitrile to the chromatographic matrix to elute molecular species of the guanine-rich oligonucleotide, wherein the guanine-rich oligonucleotide elutes in a first set of elution fractions and the quadruplex elutes in a second set of elution fractions.

A guanine-rich oligonucleotide to be separated according to the methods of the invention is an oligonucleotide comprising at least one sequence motif of three or more consecutive guanine bases. Oligonucleotides containing such sequence motifs (also referred to as G-tracts) separated by other bases have been observed to spontaneously fold into quadruplex (also referred to as G-quadruplex or tetraplex) secondary structures. See, e.g., Burge et al., Nucleic Acids Research, Vol. 34: 5402-5415, 2006 and Rhodes and Lipps, Nucleic Acids Research, Vol. 43: 8627-8637, 2015. Quadruplexes are four-stranded helical structures that are assembled from planar G-quartets that are formed from the association of four guanine bases into a cyclic arrangement stabilized by Hoogsteen hydrogen bonding. The G-quartets can stack on top of each other to form the four-stranded helical quadruplex structure. See Burge et al., 2006 and Rhodes and Lipps, 2015. Quadruplexes can be formed from intramolecular or intermolecular folding of guanine-rich oligonucleotides depending on the number of G-tracts (i.e. sequence motifs of three or more consecutive guanine bases) present in the oligonucleotides. For example, quadruplexes can be formed from the intramolecular folding of a single oligonucleotide comprising four or more G-tracts. Alternatively, quadruplexes can be formed from the intermolecular folding of two oligonucleotides comprising at least two G-tracts or four oligonucleotides comprising at least one G-tract. See Burge et al., 2006 and Rhodes and Lipps, 2015.

In certain embodiments, the guanine-rich oligonucleotide to be separated according to the methods of the invention has at least one sequence motif of three consecutive guanine bases. In other embodiments, the guanine-rich oligonucleotide has at least one sequence motif of four consecutive guanine bases. In yet other embodiments, the guanine-rich oligonucleotide has a single sequence motif of three consecutive guanine bases. In still other embodiments, the guanine-rich oligonucleotide has a single sequence motif of four consecutive guanine bases. In some embodiments, the guanine-rich oligonucleotide has a sequence of at least four consecutive guanine bases. The guanine-rich oligonucleotide to be used in the methods of the invention may contain a quadruplex-forming consensus sequence, such as those found in telomeres or certain promoter regions. For instance, in one embodiment, the guanine-rich oligonucleotide may comprise a sequence motif of TTAGGG (SEQ ID NO: 5). In another embodiment, the guanine-rich oligonucleotide may comprise a sequence motif of GGGGCC (SEQ ID NO: 6). In another embodiment, the guanine-rich oligonucleotide may comprise a sequence motif of (GₚN_{q})ₙ, where G is a guanine base, N is any nucleobase, p is at least 3, q is 1-7, and n is 1-4. In certain embodiments, p is 3 or 4.

As used herein, an oligonucleotide refers to an oligomer or polymer of nucleotides. The oligonucleotide may comprise ribonucleotides, deoxyribonucleotides, modified nucleotides, or combinations thereof. Oligonucleotides can be a few nucleotides in length up to several hundred nucleotides in length, for example, from about 10 nucleotides in length to about 300 nucleotides in length, from about 12 nucleotides in length to about 100 nucleotides in length, from about 15 nucleotides in length to about 250 nucleotides in length, from about 20 nucleotides in length to about 80 nucleotides in length, from about 15 nucleotides in length to about 30 nucleotides in length, from about 18 nucleotides in length to about 26 nucleotides in length, or from about 19 nucleotides in length to about 23 nucleotides in length. In some embodiments, the guanine-rich oligonucleotide to be purified according to the methods of the invention is about 18, 19, 20, 21, 22, 23, 24, 25, or 26 nucleotides in length. In one embodiment, the guanine-rich oligonucleotide is about 19 nucleotides in length. In another embodiment, the guanine-rich oligonucleotide is about 20 nucleotides in length. In yet another embodiment, the guanine-rich oligonucleotide is about 21 nucleotides in length. In still another embodiment, the guanine-rich oligonucleotide is about 23 nucleotides in length.

The guanine-rich oligonucleotide may be a naturally occurring oligonucleotide isolated from a cell or organism. For instance, the guanine-rich oligonucleotide may be derived from or a fragment of genomic DNA, particularly the telomere or promoter regions, or may be derived from or a fragment of messenger RNA (mRNA), particularly the 5′ or 3′ untranslated regions. In some embodiments, the guanine-rich oligonucleotide is a synthetic oligonucleotide produced by chemical synthetic methods or in vitro enzymatic methods. In some embodiments, the guanine-rich oligonucleotide can be a short hairpin RNA (shRNA), a precursor miRNA (pre-miRNA), an anti-miRNA oligonucleotide (e.g. antagomir and antimiR), or an antisense oligonucleotide. In other embodiments, the guanine-rich oligonucleotide can be one of the component strands of a double-stranded RNA molecule or RNA interference agent, such as a small interfering RNA (siRNA), a microRNA (miRNA), or a miRNA mimetic.

In certain embodiments, the guanine-rich oligonucleotide is a therapeutic oligonucleotide designed to target a gene or RNA molecule associated with a disease or disorder. For instance, in one embodiment, the guanine-rich oligonucleotide is an antisense oligonucleotide that comprises a sequence complementary to a region of a target gene or mRNA sequence having at least three or at least four consecutive cytosine bases. A first sequence is “complementary” to a second sequence if an oligonucleotide comprising the first sequence can hybridize to an oligonucleotide comprising the second sequence to form a duplex region under certain conditions. “Hybridize” or “hybridization” refers to the pairing of complementary oligonucleotides, typically via hydrogen bonding (e.g. Watson-Crick, Hoogsteen or reverse Hoogsteen hydrogen bonding) between complementary bases in the two oligonucleotides. A first sequence is considered to be fully complementary (100% complementary) to a second sequence if an oligonucleotide comprising the first sequence base pairs with an oligonucleotide comprising the second sequence over the entire length of one or both nucleotide sequences without any mismatches.

In another embodiment, the guanine-rich oligonucleotide is an antisense strand of an siRNA or other type of double-stranded RNA interference agent, wherein the antisense strand comprises a sequence that is complementary to a region of a target gene or mRNA sequence having at least three or at least four consecutive cytosine bases. In yet another embodiment, the guanine-rich oligonucleotide is a sense strand of an siRNA or other type of double-stranded RNA interference agent, wherein the sense strand comprises a sequence identical to a region of a target gene or mRNA sequence having at least three or at least four consecutive guanine bases. The strand of an siRNA or other type of double-stranded RNA interference agent comprising a region having a sequence that is complementary to a target sequence (e.g. target mRNA) is referred to as the “antisense strand.” The ‘sense strand’ refers to the strand that includes a region that is complementary to a region of the antisense strand.

The guanine-rich oligonucleotide to be purified according to the methods of the invention may comprise one or more modified nucleotides. A “modified nucleotide” refers to a nucleotide that has one or more chemical modifications to the nucleoside, nucleobase, pentose ring, or phosphate group. Such modified nucleotides can include, but are not limited to, nucleotides with 2′ sugar modifications (2′-O-methyl, 2′-methoxyethyl, 2′-fluoro, deoxynucleotides, etc.), abasic nucleotides, inverted nucleotides (3′-3′ linked nucleotides), phosphorothioate linked nucleotides, nucleotides with bicyclic sugar modifications (e.g. LNA, ENA), and nucleotides comprising base analogs (e.g. universal bases, 5-methylcytosine, pseudouracil, etc.).

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. O—C₁-C₁₀ or O—C₁-C₁₀ substituted alkyl), 2′-O-allyl (O—CH₂CH═CH₂), 2′-C-allyl, 2′-fluoro, 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy. A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA: Aminooxy (4′-CH₂—O—N(R)— 2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)— 2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into guanine-rich oligonculeotide are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the guanine-rich oligonucleotides comprise one or more 2′-fluoro modified nucleotides, 2′-O-methyl modified nucleotides, 2′-O-methoxyethyl modified nucleotides, 2′-O-allyl modified nucleotides, bicyclic nucleic acids (BNAs), or combinations thereof. In certain embodiments, the guanine-rich oligonucleotides comprise one or more 2′-fluoro modified nucleotides, 2′-O-methyl modified nucleotides, 2′-O-methoxyethyl modified nucleotides, or combinations thereof. In one particular embodiment, the guanine-rich oligonucleotides comprise one or more 2′-fluoro modified nucleotides, 2′-O-methyl modified nucleotides, deoxynucleotides, or combinations thereof. In another particular embodiment, the guanine-rich oligonucleotides comprise one or more 2′-fluoro modified nucleotides, 2′-O-methyl modified nucleotides, or combinations thereof.

The guanine-rich oligonucleotides that can be used in the methods of the invention may also comprise one or more modified intemucleotide linkages. As used herein, the term “modified intemucleotide linkage” refers to an intemucleotide linkage other than the natural 3′ to 5′ phosphodiester linkage. In some embodiments, the modified intemucleotide linkage is a phosphorous-containing intemucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified intemucleotide linkage is a 2′ to 5′ phosphodiester linkage. In other embodiments, the modified intemucleotide linkage is a non-phosphorous-containing intemucleotide linkage and thus can be referred to as a modified intemucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified intemucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified intemucleotide and intemucleoside linkages that may be incorporated into the guanine-rich oligonucleotides are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the guanine-rich oligonucleotides comprise one or more phosphorothioate intemucleotide linkages. The guanine-rich oligonucleotides may comprise 1, 2, 3, 4, 5, 6, 7, 8, or more phosphorothioate intemucleotide linkages. In some embodiments, all of the intemucleotide linkages in the guanine-rich oligonucleotides are phosphorothioate intemucleotide linkages. In other embodiments, the guanine-rich oligonucleotides can comprise one or more phosphorothioate intemucleotide linkages at the 3′-end, the 5′-end, or both the 3′- and 5′-ends. For instance, in certain embodiments, the guanine-rich oligonucleotides comprise about 1 to about 6 or more (e.g., about 1, 2, 3, 4, 5, 6 or more) consecutive phosphorothioate intemucleotide linkages at the 3′-end. In other embodiments, the guanine-rich oligonucleotides comprise about 1 to about 6 or more (e.g., about 1, 2, 3, 4, 5, 6 or more) consecutive phosphorothioate intemucleotide linkages at the 5′-end.

The guanine-rich oligonucleotides to be used in the methods of the invention can readily be made using techniques known in the art, for example, using conventional nucleic acid solid phase synthesis. The oligonucleotides can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA). The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides. The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide). The various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing oligonucleotides are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

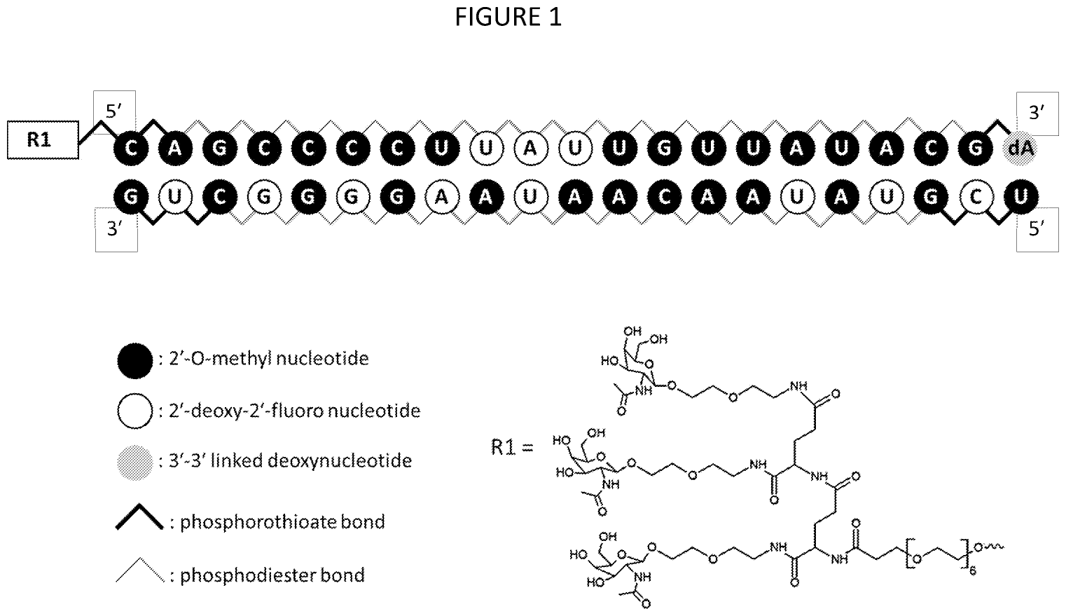

In various aspects, the guanine-rich oligonucleotide to be used in the methods of the invention comprises or consists of the sequence of 5′-UCGUAUAACAAUAAGGGGCUG-3′ (SEQ ID NO: 2). In some such embodiments, the guanine-rich oligonucleotide comprises or consists of the sequence of modified nucleotides according to the sequence of 5′-usCfsgUfaUfaacaaUfaAfgGfgGfcsUfsg-3′ (SEQ ID NO: 4), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (2‘-fluoro’) adenosine, 2′-fluoro guanosine, 2′-fluoro cytidine, and 2′-fluoro uridine, respectively; and s is a phosphorothioate linkage. In various instances, a complementary oligonucleotide of the guanine-rich oligonucleotide comprises or consists of the sequence of 5′-CAGCCCCUUAUUGUUAUACGA-3′ (SEQ ID NO: 1). In related embodiments, the complementary oligonucleotide comprises or consists of the sequence of modified nucleotides according to the sequence of 5′-csagccccuUfAfUfuguuauacgs(invdA)-3′ (SEQ ID NO: 3), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; invdA is an inverted deoxyadenosine (3′-3′ linked nucleotide), and s is a phosphorothioate linkage. In exemplary aspects, the guanine-rich oligonucleotide is the antisense strand of an siRNA and its complementary oligonucleotide is the sense strand. In various aspects, the guanine-rich oligonucleotide and its complementary oligonucleotide hybridize to form a duplex. In certain embodiments, the duplex may be olpasiran comprising a sense strand comprising the sequence of modified nucleotides according to SEQ ID NO: 3 and an antisense strand comprising the sequence of modified nucleotides according to SEQ ID NO: 4. The structure of olpasiran is shown in FIG. 1 and is further described in Example 1.

As can be appreciated by the skilled artisan, further methods of synthesizing the guanine-rich oligonucleotides will be evident to those of ordinary skill in the art. For instance, the oligonucleotides can be synthesized using enzymes in in vitro systems, such as in the methods described in Jensen and Davis, Biochemistry, Vol. 57: 1821-1832, 2018. Naturally occurring oligonucleotides can be isolated from cells or organisms using conventional methods. Custom synthesis of oligonucleotides is also available from several commercial vendors, including Dharmacon, Inc. (Lafayette, CO), AxoLabs GmbH (Kulmbach, Germany), and Ambion, Inc. (Foster City, CA).

The methods of the invention can be used to purify or separate guanine-rich oligonucleotides or quadruplex structures from one or more impurities or other molecular species in a solution. “Purify” or “purification” refers to a process that reduces the amounts of substances that are different than the target molecule (e.g. guanine-rich oligonucleotide or quadruplex) and are desirably excluded from the final composition or preparation. The term “impurity” refers to a substance having a different structure than the target molecule and the term can include a single undesired substance or a combination of several undesired substances. Impurities can include materials or reagents used in the methods to produce the guanine-rich oligonucleotides as well as fragments or other undesirable derivatives or forms of the oligonucleotides. In certain embodiments, the impurities comprise one or more oligonucleotides having a shorter length than the target guanine-rich oligonucleotide. In these and other embodiments, the impurities comprise one or more failure sequences. Failure sequences can be generated during the synthesis of the target oligonucleotide and arise from the failure of coupling reactions during the stepwise addition of a nucleotide monomer to the oligonucleotide chain. The product of an oligonucleotide synthetic reaction is often a heterogeneous mixture of oligonucleotides of varying lengths comprising the target oligonucleotide and various failure sequences having lengths shorter than the target oligonucleotide (i.e. truncated versions of the target oligonucleotide). In some embodiments, the impurities comprise one or more process-related impurities. Depending on the synthetic method to produce the guanine-rich oligonucleotide, such process-related impurities can include, but are not limited to, nucleotide monomers, protecting groups, salts, enzymes, and endotoxins.

In exemplary embodiments of the presently disclosed methods, the method separates molecular species of a guanine-rich oligonucleotide from a mixture of molecular species. As used herein, the term “molecular species” encompasses the guanine-rich oligonucleotide itself, its complementary oligonucleotide, and any and all higher order forms comprising at least one copy of the guanine-rich oligonucleotide, including, but not limited to, a quadruplex of the guanine-rich oligonucleotide, which is formed from intermolecular or intramolecular associations of the G-rich oligonucleotide(s). The term “molecular species” in various aspects encompasses the guanine-rich oligonucleotide hybridized to its complementary oligonucleotide, e.g., a duplex, as well as the guanine-rich oligonucleotide not hybridized to its complementary oligonucleotide existing in its single stranded form. In various instances, the term “molecular species” encompasses the complementary oligonucleotide in its single stranded form. In various aspects, the guanine-rich oligonucleotide is a sense strand or an antisense strand of a small interfering RNA (siRNA). Optionally, the mixture from which the guanine-rich oligonucleotide is separated comprises single-stranded molecular species and/or double-stranded molecular species. In various aspects, the mixture comprises one or more molecular species selected from the group consisting of: an antisense single strand, a sense single strand, a duplex, and a quadruplex. In exemplary aspects, at least one molecular species of the mixture is a quadruplex formed from the guanine-rich oligonucleotide. In some such embodiments, the quadruplex is formed from four guanine-rich oligonucleotides. The guanine-rich oligonucleotide is the antisense strand of an siRNA molecule in various aspects. In these and other embodiments, the siRNA duplex comprises the antisense guanine-rich strand and a sense strand that is complementary to the guanine-rich antisense strand. In exemplary instances, the mixture comprises all of the following molecular species: an antisense single strand, a sense single strand, a duplex, and a quadruplex. In some such embodiments, either the antisense strand or the sense strand is the guanine-rich oligonucleotide, the duplex comprises the antisense strand hybridized to the sense strand, and the quadruplex is formed from the strand that is the guanine-rich oligonucleotide.

In various embodiments, the method chromatographically separates molecular species of a guanine-rich oligonucleotide from a mixture of molecular species. In various aspects, the method comprises a chromatography for separating the molecular species of the mixture. In exemplary instances, the chromatography is analytical chromatography. In other exemplary instances, the chromatography is preparative chromatography. In exemplary aspects, each molecular species of the mixture is separated by way of the time at which it elutes from the matrix. In various instances, each molecular species of the mixture elutes at a time distinct from the time at which a different molecular species elutes. For example, in exemplary instances, the guanine-rich oligonucleotide elutes at a distinct time at which the quadruplex elutes. In exemplary aspects, the mixture comprises all of the following molecular species: an antisense single strand, a sense single strand, a duplex, and a quadruplex. In exemplary instances, the duplex elutes at a first time, the sense strand elutes at a second time, the antisense strand elutes at a third time, and the quadruplex elutes at a fourth time, such that each molecular species elutes at a unique time. Optionally, each molecular species elutes in a fraction separate from that of another molecular species. In exemplary aspects, the duplex elutes in a first set of elution fractions, the sense strand elutes in a second set of elution fractions, the antisense strand elutes in third set of elution fractions, and the quadruplex elutes in a fourth set of elution fractions. In various aspects, the molecular species are separated by reversed phase-high performance liquid chromatography (RP-HPLC). Reversed phased chromatography, e.g., RP-HPLC, is described in great detail in the prior art. See, for instance, Reversed Phase Chromatography: Principles and Methods, ed. AA, Amersham Biosciences, Buckinghamshire, England (1999). In various instances, the molecular species are separated by RP-HPLC (RP-HPLC). In exemplary instances, the molecular species are chromatographically separated, and the separation is characterized as having high resolution. In various aspects, the resolution of the separation of the peaks of each molecular species (e.g., the resolution of the separation between the peak of the duplex and the peak of the sense single strand) is at least or about 1.0, optionally, at least or about 1.1, at least or about 1.2, at least or about 1.3, or at least or about 1.4. In various aspects, the resolution of the separation of the peaks of each molecular species is at least or about 1.5, optionally, at least or about 1.6, at least or about 1.7, at least or about 1.8, or at least or about 1.9. Optionally, the resolution of the separation of the peaks corresponding to each molecular species (e.g., the resolution of the separation between the peak of the duplex and the peak of the sense single strand) is at least or about 2.0 (e.g., at least or about 2.1, at least or about 2.2, at least or about 2.3, at least or about 2.4. In various aspects, the resolution of the separation is at least or about 2.4. In exemplary instances, the resolution is at least or about 2.5, at least or about 3.0, or at least or about 4.0. Optionally, the resolution of the separation between the peak of the duplex and the peak of the sense strand is at least 4.0. In various aspects, the resolution is a United States Pharmacopeia (USP) resolution and may be calculated using the USP Resolution equation (Equation 1) which uses the baseline peak width calculated using lines tangent to the peak at 50% height:

$$R = \frac{2. ( Rt 2 - Rt 1 )}{1.7 ( W 2 + W 1 )} \qquad [ Equation 1 ]$$

$$wherein$$

$$R = resolution ,$$

$$Rt = retention time ,$$

$$and$$

$$W 1 + W 2 = sum of peak widths at 50 \% peak height$$

(Taken from “Empower System Suitability: Quick Reference Guide” Waters Corp. (2002))

In various aspects, the limitation of quantitation (LOQ) of the method of each molecular species is about 0.03 mg/mL to about 0.08 mg/mL, e.g., about 0.03 mg/mL, about 0.04 mg/mL, about 0.05 mg/mL, about 0.06 mg/mL, about 0.07 mg/mL, about 0.08 mg/mL, when the signal-to-noise ratio is greater than or equal to 10.0. In various instance, the LOQ is about 0.08 mg/ml when the signal-to-noise ratio is greater than or equal to 10.0.

The mixture comprising molecular species of the guanine-rich oligonucleotide can further comprise one or more impurities or contaminants, the presence of which is not desired. The mixture can include mixtures resulting from synthetic methods to produce the oligonucleotide. For example, in one embodiment the mixture is a reaction mixture from a chemical synthetic method to produce the oligonucleotide, such as a synthetic reaction mixture obtained from an automated synthesizer. In such an embodiment, the mixture may also comprise failure sequences. In another embodiment, the mixture is a mixture from an in vitro enzymatic synthetic reaction (e.g. polymerase chain reaction (PCR)). In yet another embodiment, the mixture is a cell lysate or biological sample, for example when the guanine-rich oligonucleotide is a naturally occurring oligonucleotide isolated from a cell or organism. In still another embodiment, the mixture is a solution or mixture from another purification operation, such as the eluate from a chromatographic separation.

In various aspects, the mixture comprising the molecular species of the guanine-rich oligonucleotide is prepared in a solution comprising one or more of: water, a source of acetate, a source of potassium, and sodium chloride. In various aspects, the source of acetate is ammonium acetate, sodium acetate, or potassium acetate. In various instances, the source of potassium is potassium phosphate or potassium acetate. In exemplary aspects, the solution comprises about 50 mM to about 150 mM (e.g., about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 140 mM, about 70 mM to about 140 mM, about 80 mM to about 140 mM, about 90 mM to about 140 mM, about 100 mM to about 140 mM, about 110 mM to about 140 mM, about 120 mM to about 140 mM, about 130 mM to about 140 mM) acetate or potassium. The solution in some instances comprises about 75 mM to about 100 mM (e.g., about 75 mM to about 95 mM, about 75 mM to about 90 mM about 75 mM to about 85 mM, about 75 mM to about 80 mM, about 80 mM to about 100 mM, about 85 mM to about 100 mM, about 90 mM to about 100 mM, about 95 mM to about 100 mM) of ammonium acetate, sodium acetate, or potassium acetate. In various aspects, the solution comprises potassium phosphate and sodium chloride. Without being bound to any particular theory, the presence of the potassium, sodium, and/or the ammonium in the solution stabilizes the quadruplex and/or stabilizes the guanine-rich oligonucleotide::quadruplex ratio (e.g., stabilizes the guanine-rich oligonucleotide::quadruplex equilibrium) so that these molecular species may be better chromatographically separated. In various aspects, the mixture is prepared in water, optionally, purified, deionized water

Once the solution comprising the mixture of molecular species is prepared, it is applied to a chromatographic matrix comprising a hydrophobic ligand. Optionally, the chromatographic matrix is a reverse-phased chromatography matrix comprising hydrophobic ligands chemically grafted to a porous, insoluble beaded matrix. In various instances, the matrix is chemically and mechanically stable. Optionally, the matrix comprises silica or a synthetic organic polymer (e.g., polystyrene). In various aspects, the chromatographic matrix is housed in a chromatographic column having an internal diameter of 2.1 mm and/or a column length of about 50 mm. Optionally, the matrix comprises 1.7 ethylene bridged hybrid (BEH) particles to which the hydrophobic ligand is attached. In various instances, each particle comprises a 300 Å pore and/or has particle diameter of about 3.5 μm. The hydrophobic ligand of the matrix, in various aspects, comprises C4 alkyl chains, C6 alkyl chains, or C8 alkyl chains. In certain aspects, the ligand comprises C4 alkyl chains. Suitable chromatographic matrices are commercially available, including, e.g., the Watersm BEH columns (SKU 186004498; Waters Corporation, Milford, MA) and other similar columns having C4, C6 or C8 alkyl chains, e.g., Hypersil GOLD™ C4 HPLC Columns (ThermoFisher Scientific, Waltham, MA), Polar-RP HPLC Columns (Hawach Scientific, Xi'an City, Shaanxi Province, PR China), AdvanceBio RP-mAb columns (Agilent Technologies, Inc., Santa Clara, CA).

After the mixture is applied to the chromatographic matrix, a mobile phase is applied to the chromatographic matrix. In exemplary aspects, the mobile phase comprises a gradient of acetate and a gradient of acetonitrile. In various instance the gradient of acetate is made with an acetate stock solution comprising about 50 mM to about 150 mM acetate, e.g., about 50 mM to about 140 mM, about 50 mM to about 130 mM, about 50 mM to about 120 mM, about 50 mM to about 110 mM, about 50 mM to about 100 mM, about 50 mM to about 90 mM, about 50 mM to about 80 mM, about 50 mM to about 70 mM, about 50 mM to about 60 mM, about 60 mM to about 140 mM, about 70 mM to about 140 mM, about 80 mM to about 140 mM, about 90 mM to about 140 mM, about 100 mM to about 140 mM, about 110 mM to about 140 mM, about 120 mM to about 140 mM, about 130 mM to about 140 mM acetate. Optionally, the acetate stock solution comprises about 70 mM to about 80 mM acetate, optionally, about 75 mM acetate or about 90 mM to about 110 mM acetate, optionally, about 100 mM acetate. In various aspects, the acetate is ammonium acetate, sodium acetate or potassium acetate. Other counterions are contemplated herein. In certain embodiments, the acetate is ammonium acetate. The pH of the acetate stock solution is about 6.5 to about 7.0 (e.g., 6.5, 6.6, 6.7, 6.8, 6.9, 7.0) in various instances. For example, the pH of the acetate stock solution is about 6.7 or about 6.8 to about 7.0. In various instances, the acetate stock solution is 75 mM ammonium acetate in water having a pH of 6.7±0.1. In exemplary aspects, the gradient of acetonitrile is made with an acetonitrile stock solution and the acetonitrile stock solution is 100% acetonitrile. In exemplary aspects, the mobile phase comprises a decreasing concentration gradient of the acetate and an increasing concentration gradient of acetonitrile. The gradient of acetate, in various aspects, starts with a maximum concentration and gradually decreases to a minimum concentration over a first time period. The first time period is about 18 to about 19 minutes in exemplary instances. In alternative instances, the first time period is about 22 minutes to about 26 minutes. After the first time period, the acetate concentration in the mobile phase increases to the maximum concentration of acetate, in exemplary aspects. In various instances, the acetate concentration in the mobile phase increases to the maximum concentration of acetate about 0.1 to about 3 minutes after the gradient reaches the minimum concentration of acetate. In various instances, the gradient of acetonitrile starts with a minimum concentration and gradually increases to a maximum concentration over the first time period. Optionally, after the first time period, the acetonitrile concentration in the mobile phase decreases to the minimum concentration of acetonitrile. For example, the acetonitrile concentration in the mobile phase decreases to the minimum concentration about 0.1 to about 3 minutes after the gradient of acetonitrile reaches the maximum concentration of acetonitrile. In various instances, the method comprises applying the mobile phase to the chromatographic matrix according to the following conditions:

| Time (min) | Acetate (%) | Acetonitrile |
|---|---|---|
| 0 | 93 | 7 |
| 5 | 88 | 12 |
| 8 | 88 | 12 |
| 11 | 86 | 14 |
| 18 | 70 | 30 |
| 19 | 70 | 30 |
| 21 | 93 | 7 |
| 26 | 93 | 7 |

In alternative instances, the method comprises applying the mobile phase to the chromatographic matrix according to the following conditions:

| Time (min) | Acetate (%) | Acetonitrile (%) |
|---|---|---|
| 0 | 92 | 8 |
| 2 | 90 | 10 |
| 18 | 86 | 14 |
| 26 | 70 | 30 |
| 26.1 | 92 | 8 |
| 30 | 92 | 8 |

In alternative or additional aspects, the method comprises applying the mobile phase to the chromatographic matrix according to the following conditions:

| Time (min) | Acetate (%) | % Acetonitrile |
|---|---|---|
| 0.0 | 92 | 8 |
| 2.0 | 90 | 10 |
| 18.0 | 86 | 14 |
| 22.0 | 78 | 22 |
| 22.1 | 20 | 80 |
| 24.0 | 20 | 80 |
| 24.1 | 92 | 8 |
| 30.0 | 92 | 8 |

In some embodiments of the methods of the invention, the mobile phase does not comprise a cationic ion pairing agent. Ion pairing agents are believed to bind to the solute molecules through ionic interactions to increase the hydrophobicity of the solute molecule and change selectivity. For oligonucleotides, which are highly negatively charged, cationic ion pairing agents are often included and even required in the mobile phase to achieve any separation by reversed-phase chromatography. As described in the Examples, the methods of the invention do not require cationic ion pairing agents in the mobile phase and are preferably omitted from the mobile phase to achieve the high-resolution separation of the molecular species of the guanine-rich oligonucleotide. Cationic ion pairing agents are known in the art and include, but are not limited to, a trialkylammonium species, hexylammonium acetate (HAA), tetramethylammonium chloride, tetrabutylammonium chloride, triethylammonium acetate (TEAA), triethylamine (TEA), tert-butylamine, propylamine, diisopropylethylamine (DIPEA), dimethyl n-butylamine (DMBA).

The mobile phase in various aspects is applied to the chromatographic matrix for a total run time of at least about 25 minutes and less than 40 minutes. In various aspects, the total run time is less than 35 minutes, optionally, less than or equal to 30 minutes. Optionally, the total run time is about 22 minutes to about 26 minutes.

The separation on the chromatographic matrix can be carried out at ambient temperature. For instance, in some embodiments, the separation on the chromatographic matrix is conducted at a temperature of about 20° C. to about 35° C. In other embodiments, the separation on the chromatographic matrix is conducted at a temperature of about 30° C. The formation and stability of quadruplex secondary structures and the equilibrium between a guanine-rich oligonucleotide and the quadruplex can be impacted by temperature. Accordingly, in some embodiments, the separation on the chromatographic matrix is conducted at a temperature of less than 20° C., less than 15° C., or less than 10° C., such as at about 8° C.

Suitable flow rates at which the mobile phase can be applied to the chromatographic matrix include, but are not limited to, about 0.5 mL/min to about 1.5 mL/min. In certain embodiments, the mobile phase is applied to the chromatographic matrix at a flow rate of about 0.5 mL/min to about 1.0 mL/min. In other embodiments, the mobile phase is applied to the chromatographic matrix at a flow rate of about 0.6 mL/min to about 0.9 mL/min. In still other embodiments, the mobile phase is applied to the chromatographic matrix at a flow rate of about 0.7 mL/min to about 0.8 mL/min. In one embodiment, the mobile phase is applied to the chromatographic matrix at a flow rate of about 0.7 mL/min or 0.8 mL/min. A person of ordinary skill in the art can determine other appropriate flow rates for the mobile phase depending on the pore size of the chromatographic matrix and the bed volume of the column to maintain acceptable pressure levels.

In various aspects, the method comprises applying the mobile phase to the chromatographic matrix to elute molecular species of the guanine-rich oligonucleotide present in the mixture. In various instances, at least the guanine-rich oligonucleotide elutes at a time distinct from the time the quadruplex elutes. In various aspects, each molecular species of the mixture elutes at a time distinct from the time at which another molecular species elutes. In various instances, each molecular species of the mixture elutes in a fraction separate from that of another molecular species. In various aspects, the guanine-rich oligonucleotide elutes in a first set of elution fractions and the quadruplex elutes in a second set of elution fractions. For example, in embodiments in which the mixture comprises the guanine-rich oligonucleotide, a complementary oligonucleotide to the guanine-rich oligonucleotide, a duplex comprising the guanine-rich oligonucleotide hybridized to the complementary oligonucleotide, and a quadruplex formed form the guanine-rich oligonucleotide, the guanine-rich oligonucleotide compound elutes separately from the quadruplex, which elutes separately from the duplex and the complementary oligonucleotide. In some such embodiments, the duplex elutes in a first set of elution fractions, the complementary oligonucleotide elutes in a second set of elution fractions, the guanine-rich oligonucleotide elutes in a third set of elution fractions, and the quadruplex elutes in a fourth set of elution fractions. In various aspects, the method achieves high resolution separation of each molecular species of the guanine-rich oligonucleotide.

In various aspects of the present disclosure, elution fractions are collected as the mixture comprising the molecular species is moved through the chromatographic matrix with the mobile phase described herein. In various aspects, the method further comprises collecting the elution fractions into separate containers over a time period. In various aspects, the method comprises monitoring elution of molecular species using an ultraviolet detector. The oligonucleotide content in the fractions can be monitored using UV absorption at 260 nm or at 295 nm. As shown by the chromatograms in the figures, when the chromatography is operated according to the methods of the invention, the single-stranded guanine-rich oligonucleotide elutes from the chromatographic matrix prior to the quadruplex, thus enabling the collection of separate sets of fractions for the single-stranded guanine-rich oligonucleotide and for the quadruplex. Samples from the elution fractions can be analyzed by gel electrophoresis, capillary electrophoresis, ion-pairing reversed phase liquid chromatography-mass spectrometry, analytical ion exchange chromatography, and/or native mass spectrometry to verify the enrichment of the fractions for the single-stranded guanine-rich oligonucleotide and the quadruplex.

In certain embodiments of the methods of the invention, the elution fraction or set of elution fractions comprising the single-stranded guanine-rich oligonucleotide can be isolated and optionally pooled for further processing. For instance, the elution fraction(s) containing the guanine-rich oligonucleotide may be subject to one or more further purification steps, such as affinity separation (e.g. nucleic acid hybridization using sequence-specific reagents), ion exchange chromatography steps (e.g. using different stationary phases), additional reverse-phase chromatography, or size-exclusion chromatography (e.g. with a desalting column). In these and other embodiments, the elution fraction(s) containing the guanine-rich oligonucleotide may be subject to other reactions to modify the structure of the guanine-rich oligonucleotide. For example, in embodiments in which the guanine-rich oligonucleotide is a therapeutic molecule (e.g. antisense oligonucleotide) or component of a therapeutic molecule (e.g. double-stranded RNA interference agent, such as siRNA), the purified guanine-rich oligonucleotide in the elution fraction(s) may be subject to a conjugation reaction to covalently attach a targeting ligand, such as a carbohydrate-containing ligand, cholesterol, antibody, and the like, to the oligonucleotide. In other embodiments, the purified guanine-rich oligonucleotide in the elution fraction(s) may be encapsulated in exosomes, liposomes, or other type of lipid nanoparticle or formulated in a pharmaceutical composition with a pharmaceutically acceptable excipient for administration to patients for therapeutic purposes. In embodiments in which the guanine-rich oligonucleotide is a component of a double-stranded RNA interference agent (e.g. either the sense strand or antisense strand of an siRNA molecule), the purified guanine-rich oligonucleotide in the elution fraction(s) may be subject to an annealing reaction to hybridize the guanine-rich oligonucleotide with its complementary strand to form the double-strand RNA interference agent. In some embodiments of the methods of the invention, the elution fraction or set of elution fractions comprising the quadruplex can be isolated and optionally pooled for further processing. The quadruplex can be used as an intact structure in subsequent assays or analyses to study and evaluate the function of the quadruplex structure in various systems.

In exemplary aspects of the presently disclosed methods, the method is a non-denaturing method or does not comprise any denaturing steps, such that any quadruplex, duplex, or other higher order structures of the guanine-rich oligonucleotides present in the mixture of molecular species would be subject to denaturing conditions. The denaturing conditions can include denaturing by elevations in temperature, elevations in pH, exposure to chaotropic agents, exposure to organic agents other than those in the mobile phase, or combinations of any of these conditions. Thus, in exemplary aspects, the method does not include denaturing by heating the chromatographic matrix or conducting the separation at elevated temperature sufficient to disrupt the hydrogen bonding interactions among the guanine bases forming the G-quartets. For instance, the temperature of the chromatographic matrix is not heated to a temperature above 45° C., such as from about 45° C. to about 95° C., from about 55° C. to about 85° C., or from about 65° C. to about 75° C. In other embodiments, the mobile phase does not have a pH in the strongly alkaline range, which can denature the quadruplex and other higher order structures of the guanine-rich oligonucleotide. For example, the pH of the mobile phase is below a pH of about 8.0. In certain embodiments, the mobile phase used in the methods of the invention does not comprise a chaotropic agent. A chaotropic agent is a substance that disrupts the hydrogen bonding network among water molecules and can reduce the order in the structure of macromolecules by affecting intramolecular interactions mediated by non-covalent forces, such as hydrogen bonding, van der Waals forces, and hydrophobic interactions. Chaotropic agents include, but are not limited to, guanidinium chloride and other guanidinium salts, lithium acetate or lithium perchlorate, magnesium chloride, phenol, sodium dodecyl sulfate, urea, thiourea, and a thiocyanate salt (e.g. sodium thiocyanate, ammonium thiocyanate, or potassium thiocyanate).

The methods of the invention provide substantially pure preparations of the guanine-rich oligonucleotide. For instance, in some embodiments, the purity of the guanine-rich oligonucleotide in elution fractions from the chromatographic matrix is at least 75%, at least 80%, at least 85%, at least 90%, at least 95%, at least 96%, at least 97%, at least 98%, or at least 99%. In certain embodiments, the purity of the guanine-rich oligonucleotide in elution fractions from the chromatographic matrix is at least 85%. In other embodiments, the purity of the guanine-rich oligonucleotide in elution fractions from the chromatographic matrix is at least 88%. In still other embodiments, the purity of the guanine-rich oligonucleotide in elution fractions from the chromatographic matrix is at least 90%. Methods of detecting and quantitating oligonucleotides are known to those of skill in the art and can include analytical ion exchange methods and ion-pairing reversed phase liquid chromatography-mass spectrometry methods and, such as those described in the examples.

Advantageously, the methods of the present disclosure may be used to achieve high resolution separation for the guanine-rich oligonucleotide, its complementary strand, the quadruplex and the duplex comprising the guanine-rich oligonucleotide and its complementary strand. The presently disclosed methods are thus useful for determining the purity of a sample comprising a guanine-rich oligonucleotide, a guanine-rice oligonucleotide drug substance or drug product. Accordingly, the present invention provides a method of determining the purity of a sample comprising a guanine-rich oligonucleotide drug substance or drug product. In exemplary embodiments, the method comprises separating molecular species of the guanine-rich oligonucleotide in accordance with the presently disclosed methods of separating molecular species of the guanine-rich oligonucleotide. In various aspects, the sample is an in-process sample and the method is used as part of an in-process control assay or as an assay for ensuring the manufacture of the G-rich oligonucleotide is being carried out without substantial impurities. In various instances, the sample is a lot sample and the method is used as part of a lot release assay.

In various aspects, the sample is a stressed sample or a sample that has been exposed to one or more stresses, and the method is a stability assay. Accordingly, the present invention provides a method of testing stability of a guanine-rich oligonucleotide drug substance or drug product, comprising applying stress to a sample comprising the guanine-rich oligonucleotide drug substance or drug product and determining the purity of the sample according to a method of the present disclosure. In exemplary instances, the presence of impurities in the sample after the one or more stresses indicates instability of the G-rich oligonucleotide under the one or more stresses. In exemplary aspects, the stress that has been applied to the sample is an (A) exposure to visible light, ultra-violet (UV) light, heat, air/oxygen, freeze/thaw cycle, shaking/agitation, chemicals and materials (e.g., metals, metal ions, chaeotropic salts, detergents, preservatives, organic solvents, plastics), molecules and cells (e.g., immune cells), or (B) change in pH (e.g., a change of greater than 1.0, 1.5, or 2.0), pressure, temperature, osmolality, salinity, or (C) long-term storage. The change in temperature in some aspects, is a change of at least or about 1 degree C., at least or about 2 degrees C., at least or about 3 degrees C., at least or about 4 degrees C., at least or about 5 degrees C., or more. The methods of the present disclosure are not limited to any particular types of stresses. In exemplary aspects, the stress is an exposure to elevated temperatures to, e.g., 25 degrees C., 40 degrees C., 50 degrees C. optionally, in formulation. In exemplary instances, such exposure to elevated temperatures mimics an accelerated stress program. In some aspects, the stress is exposure to visible and/or ultra-violet light; oxidizing reagents (e.g., hydrogen peroxide); air/oxygen, freeze/thaw cycle, shaking, long-term storage in formulation under the intended product storage conditions; mildly acidic pH (e.g., pH of 3-4) or elevated pH (e.g., pH of 8-9) simulate exposure to some purification conditions/steps. In some aspects, the stress is a change in pH of greater than 1.0, 1.5, 2.0 or 3.0. In exemplary aspects, the stress is an exposure to ultra-violet light, heat, air, freeze/thaw cycle, shaking, long-term storage, change in pH, or change in temperature, optionally, wherein the change in pH is greater than about 1.0 or greater than about 2.0, optionally, wherein the change in temperature is greater than or about 2 degrees Celsius or greater than or about 5 degrees Celsius.

The following examples, including the experiments conducted and the results achieved, are provided for illustrative purposes only and are not to be construed as limiting the scope of the appended claims.

### EXAMPLES

### Example 1

This example describes several initial studies which evaluated various parameters in an RP-HPLC for separating the molecular species of a G-rich oligonucleotide.

Unless stated otherwise, olpasiran, an siRNA designed to decrease the production of lipoprotein(a) (Lp(a)) by targeting mRNA transcribed from the LPA gene, was used as an exemplary oligonucleotide compound. The antisense strand of olpasiran is a G-rich oligonucleotide comprising a stretch of four consecutive guanine bases located near its 3′ end. This G-rich antisense oligonucleotide pairs with the sense strand to form the siRNA duplex. Four antisense strands can associate to form a single quadruplex structure via the stretch of guanine nucleotides in each strand. Each strand is 21 nucleotides long and contains nucleotides with chemical modifications. A targeting ligand comprising N-acetylgalactosamine is linked to the 5′ end of the sense strand for selective liver targeting. The structure of olpasiran is provided in FIG. 1.

In chromatographic separations, quadruplex can co-elute with the duplex thereby complicating the quantification of the separate molecular species. Separation of the sense strand and antisense strand can also be challenging. Thus, several initial studies were carried out to identify methods for chromatographically separating the quadruplex from the duplex and antisense strand, as well as methods which could additionally achieve chromatographic separation of the duplex and sense strand and sense strand from antisense strand for the separation of all four molecular species (e.g. quadruplex, duplex, antisense strand, and sense strand).

### Study 1

In a first study, samples comprising the duplex, quadruplex, sense strand and antisense strand of olpasiran were applied to an Agilent AdvanceBio Oligonucleotide HPH—C18 column (2.1 mm×150 mm×2.7 μm), which column was maintained at 8° C., for reversed phase-high-performance liquid chromatography (RP-HPLC). Gradient elution was carried out with decreasing concentrations of 20 mM hexylammonium acetate (HAA)+2% acetonitrile (ACN)+5% methanol (Mobile Phase A; MP A) and increasing concentrations of 20 mM HAA+ 82% ACN (Mobile Phase B; MP B). HAA is a cationic ion pairing agent. The details of the gradient mobile phase are set forth in Table 1.

TABLE 1

| Time (min) | % MP A | % MP B |
|---|---|---|
| 0.0 | 80 | 20 |
| 1.0 | 80 | 20 |
| 31.0 | 35 | 65 |

The column flow rate was set at 0.25 ml/min.

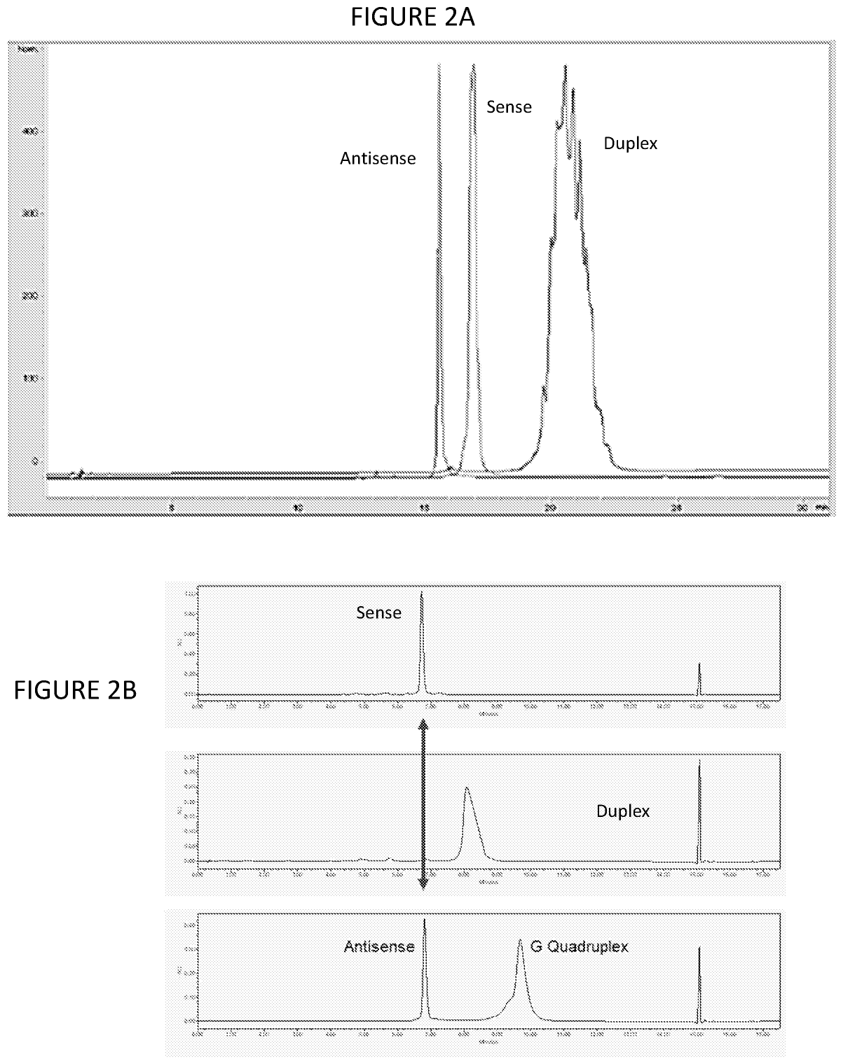

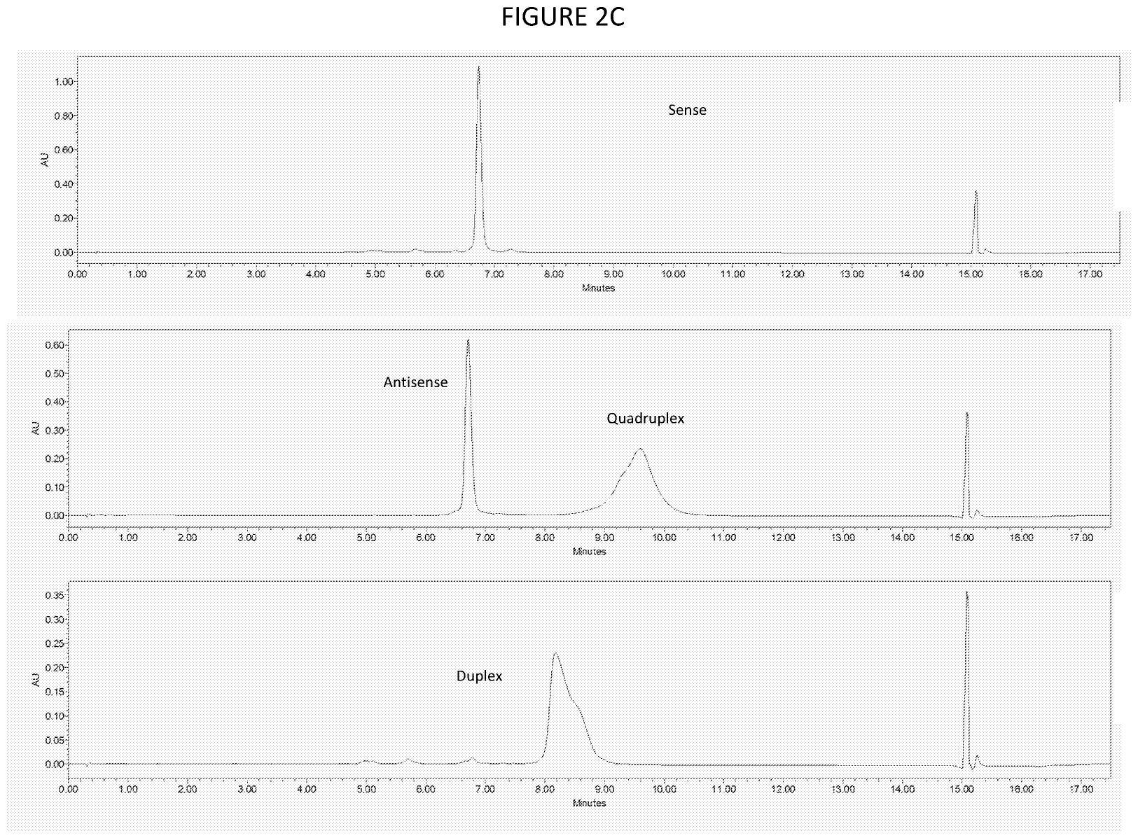

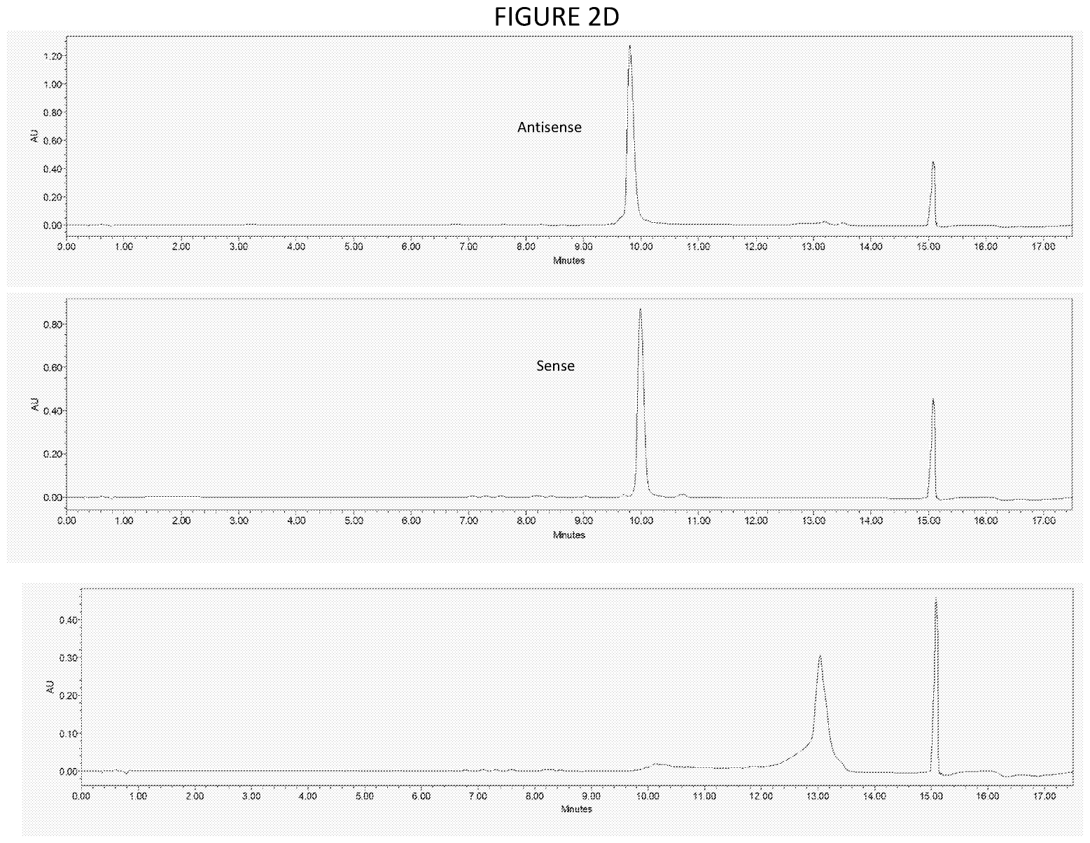

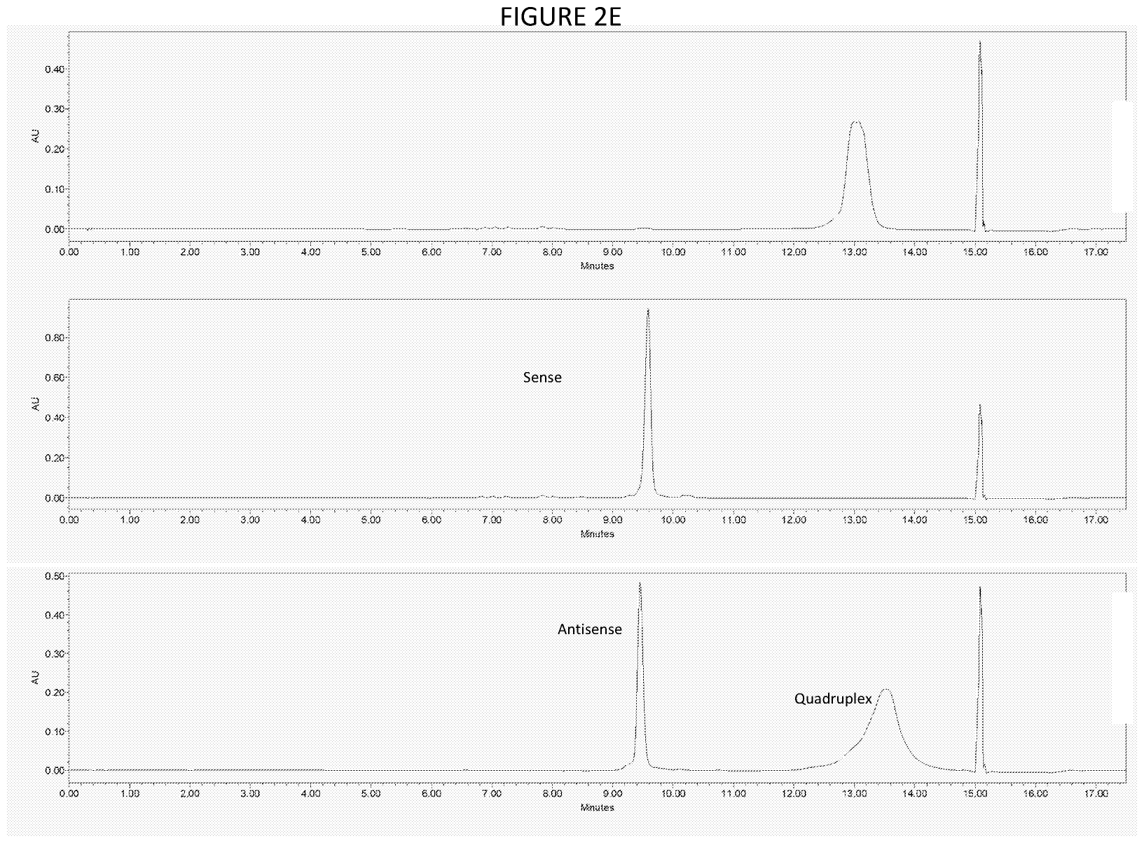

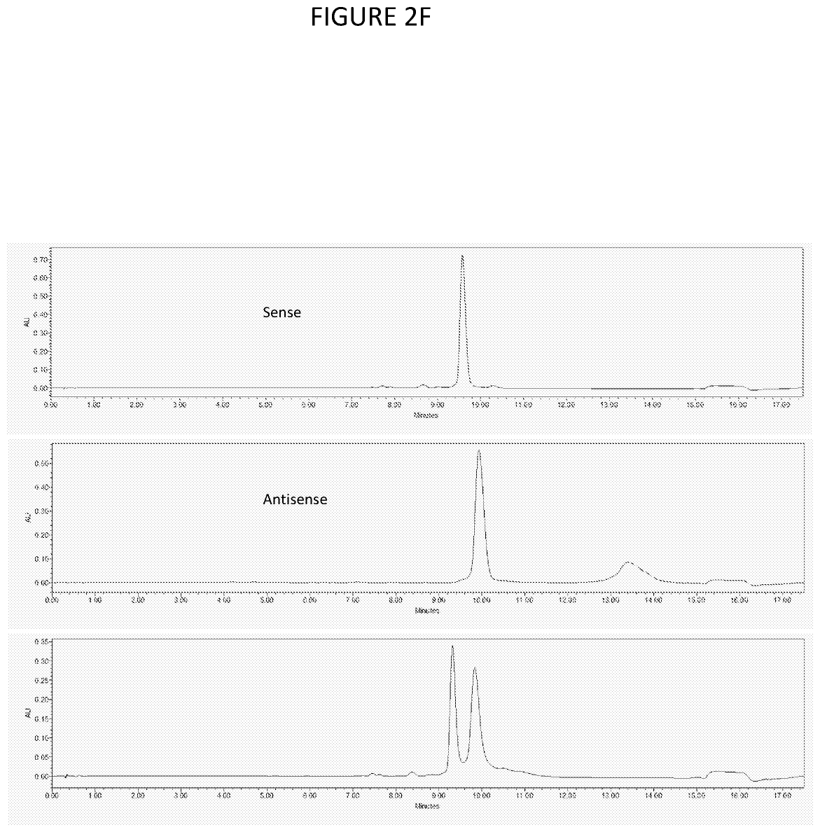

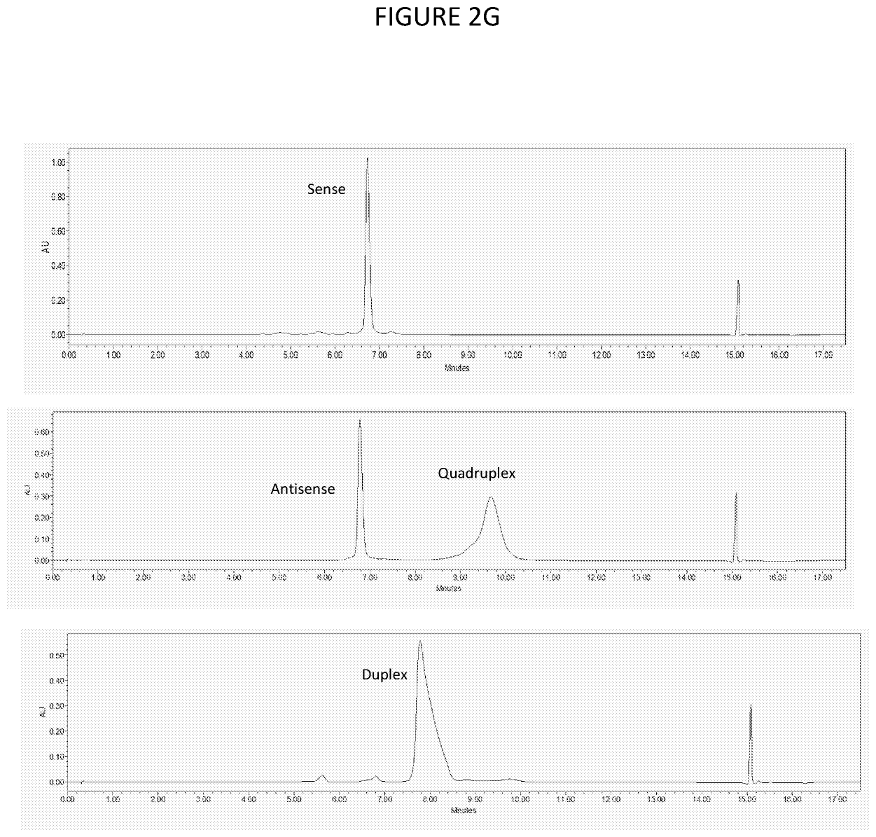

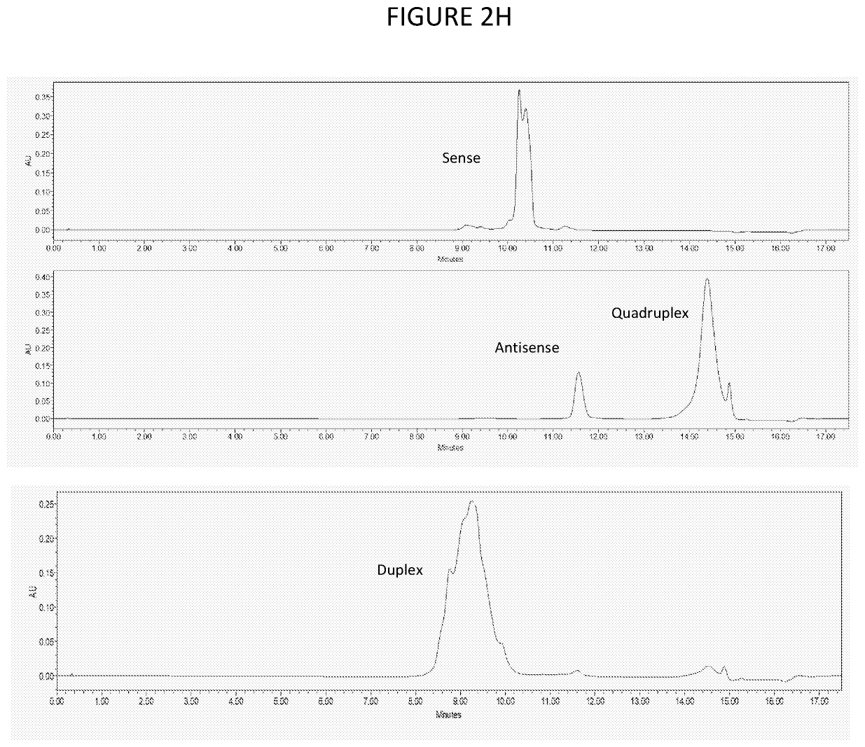

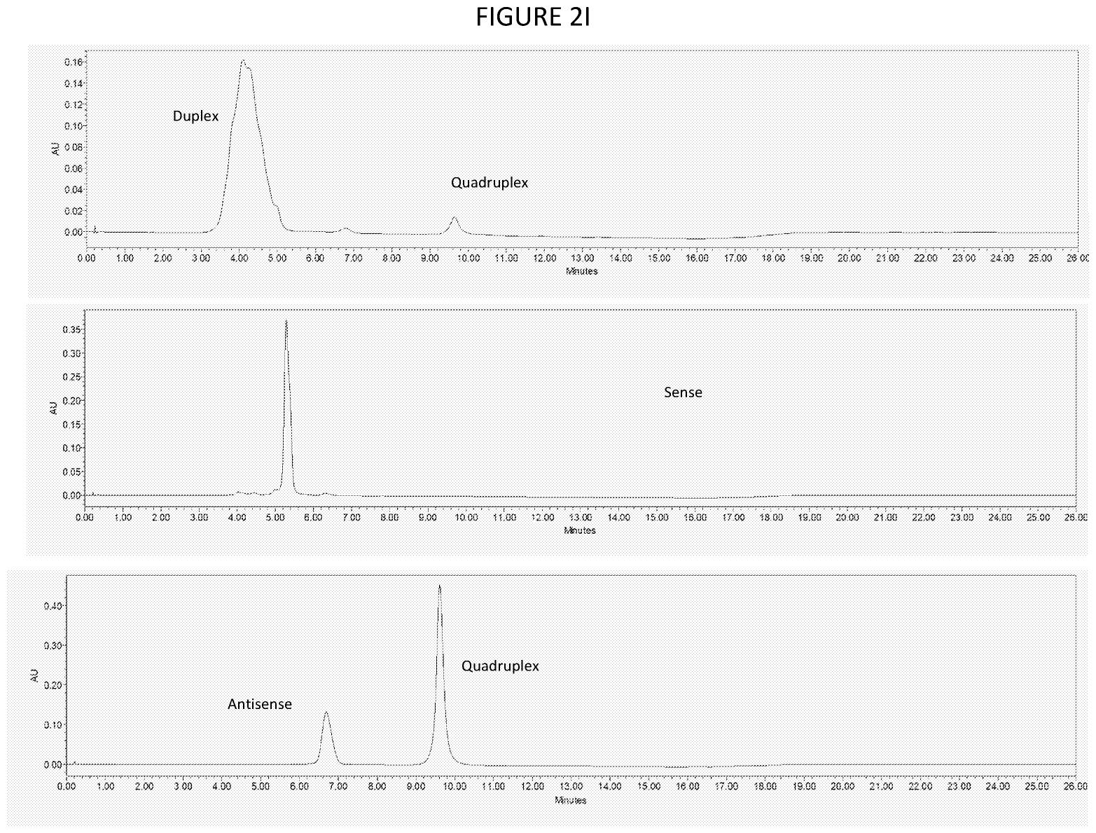

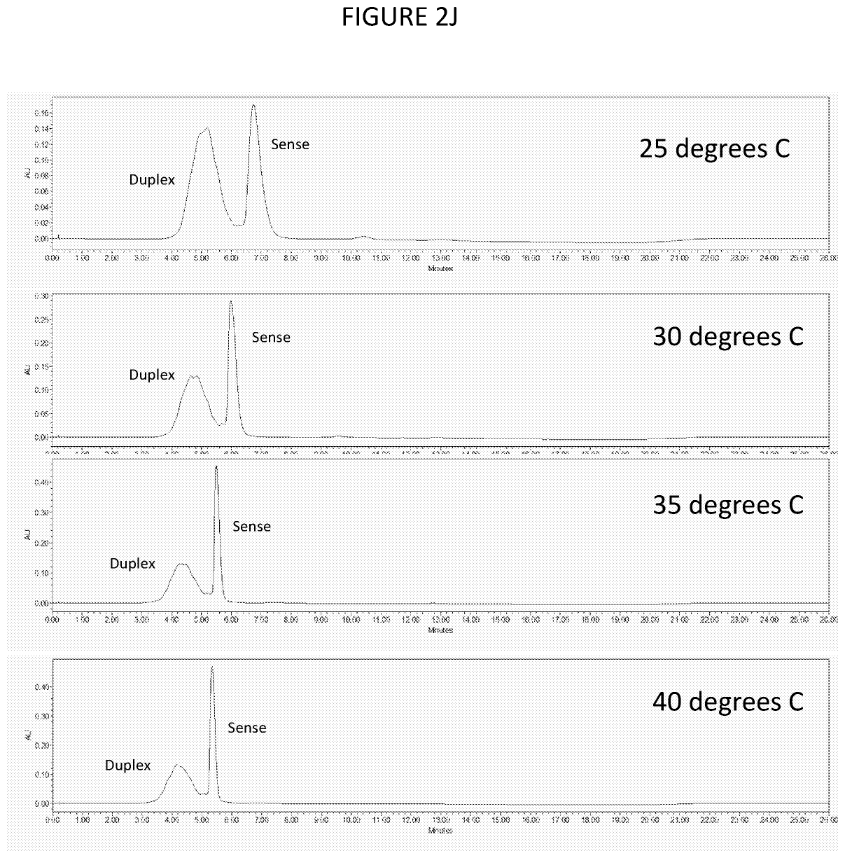

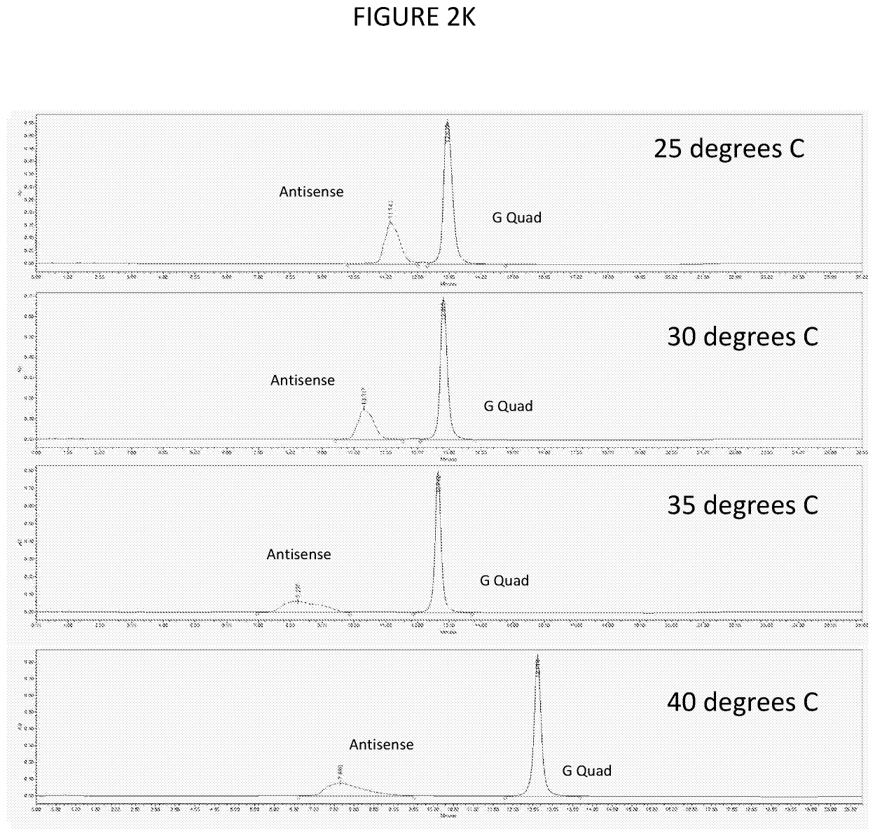

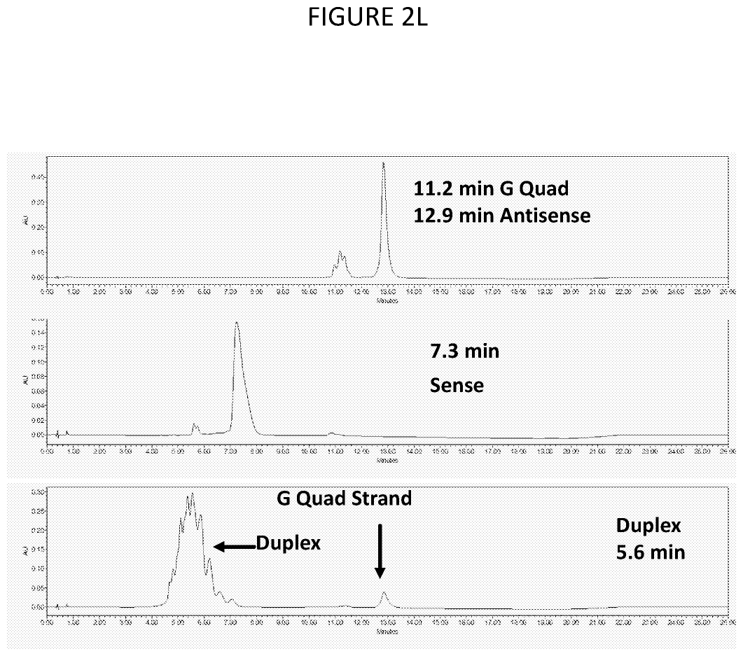

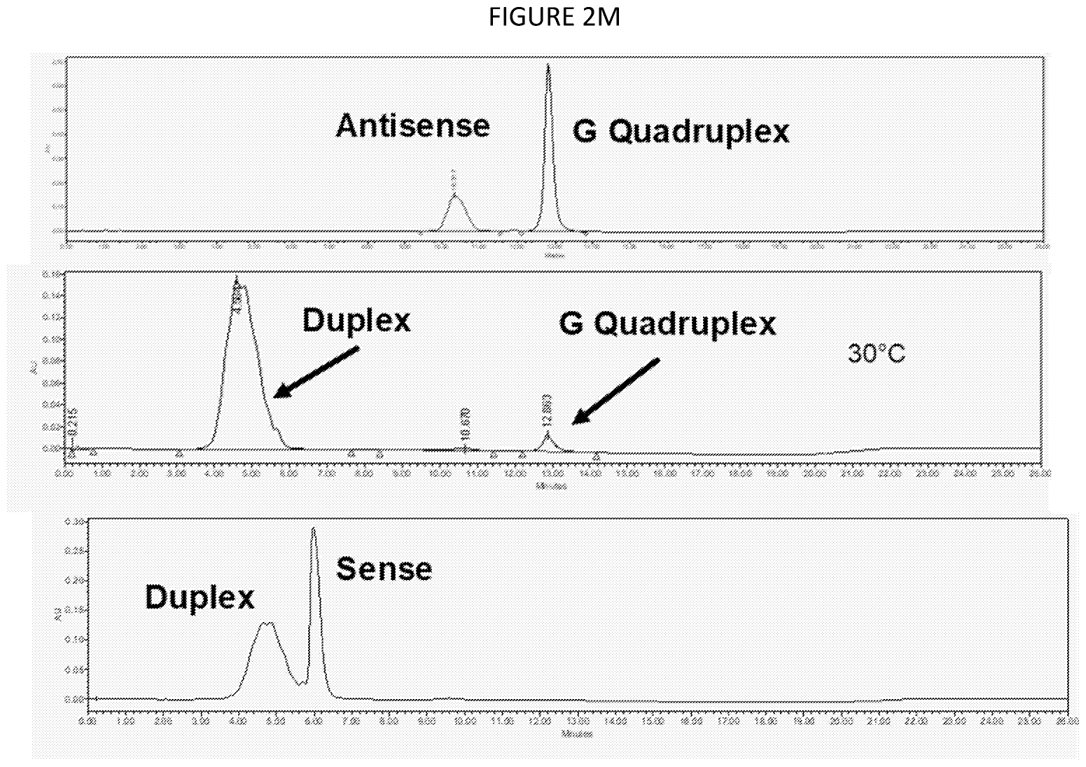

An exemplary chromatogram is shown in FIG. 2A. As shown in this figure, the antisense and sense strands were separated from the duplex with some resolution. However, this method was unable to separate or quantify the quadruplex as the quadruplex peak overlapped with the duplex peak.

### Study 2

In another study, ion pairing RP-HPLC (IP—RP-HPLC) was carried out using a Waters Xbridge BEH C4 column (2.1×50 mm, 300 Å, 3.5 μM) maintained at 35° C. After a sample comprising either olparsiran duplex, the sense strand of olpasiran, or the antisense strand of olpasiran was applied to the column, gradient elution was carried out with decreasing concentrations of 95 mM hexafluoro-isopropanol (HFIP)/8 mM Triethylamine (TEA)/24 mM tert-butylamine (Mobile Phase A; MP A) and increasing concentrations of ACN (Mobile Phase B; MP B). TEA and tert-butylamine are considered as cationic ion pairing agents. The details of the gradient mobile phase are set forth in Table 2. The column flow rate was set at 0.5 ml/min; UV monitor at 260 nm, column temperature 35° C.

TABLE 2

| Time (min) | % MP A | % MP B |
|---|---|---|
| 0.00 | 100 | 0 |
| 14.00 | 83 | 17 |
| 14.25 | 20 | 80 |
| 15.24 | 20 | 80 |
| 16.50 | 100 | 0 |

An exemplary chromatogram is provided in FIG. 2B. As shown in this figure, this method successfully separated the quadruplex from the antisense strand. However, this method failed to separate the sense and antisense strands as the retention time for each of these species is identical.

### Studies 3A-3E

Further studies were carried out to analyze the effect of the gradient elution and the components of the mobile phase with the goal of achieving high resolution separation of the antisense and sense strands. Without being bound to a particular theory, the antisense strand of olpasiran equilibrates between two molecular species: antisense single strand and quadruplex, and successful chromatographic separation of these two molecular species depends on reaching a stable state of equilibrium, which, in turn, depends on the components and ionic strength, among other characteristics, of the solution in which the molecular species are present. One goal of these studies was to determine conditions that stabilize the equilibrium.

### Study 3A

In one study (Study 3A), the mobile phase of Study 2 was modified to a mobile phase comprising HFIP, TEA and one of the following alkylamines to replace of the tert-butylamine used in Study 2: (i) propylamine, (ii) diisopropylethylamine (DIPEA), or (iii) dimethyl n-butylamine (DMBA). Each of these alkylamines, like TEA, acts as a cationic ion pairing agent. The details of each MP A of the mobile phase are set forth in Table 3. In (iv), MP A was the same as (ii) except that the concentration of HFIP was lowered to 25 mM. In (v), the mobile phase was identical to that of Study 2 except that no tert-butylamine or any other alkylamine was included.

In each instance, IP—RP-HPLC was carried out using a Waters Xbridge BEH C4 column (2.1×50 mm, 300 Å, 3.5 μM) maintained at 35° C. After a sample comprising duplex, sense strand, or antisense strand of olpasiran was applied to the column, gradient elution was carried out with decreasing concentrations of MP A and increasing concentrations of acetonitrile (MP B). The conditions for each gradient elution were as described in Table 2.

TABLE 3

| | HFIP | TEA | | Flow rate | |
|---|---|---|---|---|---|
| | (mM) | (mM) | Alkylamine | (ml/min) | FIG. |
| i | 95 | 8 | 8 mM Propylamine | 0.5 | 2C |
| ii | 95 | 0 | 8 mM DIPEA | 0.5 | 2D |
| iii | 95 | 0 | 8 mM DMBA | 0.5 | 2E |
| iv | 25 | 0 | 8 mM DIPEA | 0.5 | 2F |
| v | 95 | 8 | 0 mM alkyl amine | 0.3 | 2G |

As shown in FIGS. 2C-2E and 2G, each mobile phase described in Table 3 led to poor separation of the antisense and sense strands. As shown in FIG. 2F, the reduced HFIP concentration in the presence of DIPEA led to an increased basicity of the mobile phase, which denatured the duplex into the component sense and antisense strands. These results were surprising, given that the mobile phase comprised one or two cationic ion pairing agents, which are known as required components of the mobile phase when purifying olignonucleotides using a hydrophobic stationary phase and their inclusion has been suggested for increasing the chance of achieving a complete resolution of the sample components. See, e.g., Reversed Phase Chromatography: Principles and Methods, ed. AA, Amersham Biosciences, Buckinghamshire, England (1999).

### Study 3B

In this study, a different ion pairing agent, triethylammonium acetate (TEAA), in the mobile phase was evaluated at a much higher concentration than those used in previous studies (100 mM TEAA vs. 8 mM TEA or alkylamine used in Study 2 and Study 3A, for example). IP—RP-HPLC was carried out using a Waters Xbridge BEH C4 column (2.1×50 mm, 300 Å, 3.5 μM) maintained at 40° C. After a sample comprising duplex, sense strand, or antisense strand of olpasiran was applied to the column, gradient elution was carried out with decreasing concentrations of 100 mM TEAA/ACN (pH 7) (MP A) and increasing concentrations of ACN (MP B). The details of the gradient mobile phase are set forth in Table 4. The column flow rate was set at 0.8 ml/min. The elution was monitored using a UV monitor at 260 nm. The column temperature was 40° C.

TABLE 4

| Time | % MP A | % MP B |
|---|---|---|
| 0 | 93 | 7 |
| 5 | 88 | 12 |
| 8 | 88 | 12 |
| 11 | 86 | 14 |
| 18 | 70 | 30 |
| 19 | 70 | 30 |
| 21 | 93 | 7 |
| 26 | 93 | 7 |

The results showed no separation of the quadruplex or between single strands. Thus, the mobile phase comprising an increased concentration of a cationic ion pairing agent did not improve the separation of the molecular species. The lack of improvement in resolution was surprising given the increased concentration of the ion pairing agent.

### Study 3C

In Study 3C, size exclusion chromatography was carried out using a Water Acquity BEH SEC column (4.6 mm×150 mm, 200 Å, 1.7 μm). Two mobile phases utilizing isocratic gradients were employed. The column temperature was 30 degrees C. A mobile phase comprising 5% ACN+ammonium acetate (pH 7) with a flow rate of 0.5 ml/min was compared to 5% ACN+sodium phosphate with a 0.8 ml/min flow rate. Elution was monitored with a UV monitor at 260 nm.

Using a mobile phase comprising 5% ACN+ammonium acetate (pH 7), the quadruplex eluted at 1.49 min, the antisense eluted at 1.81 min, the sense strand eluted at 1.76 min and the duplex eluted at 1.67 min. Using a mobile phase comprising 5% ACN+sodium phosphate, the quadruplex eluted at 2.45 min, the antisense eluted at 2.97 min, the sense strand eluted at 2.85 min and the duplex eluted at 2.76 min. Although some separation of the four different molecular species was obtained using size exclusion chromatography, the elution of each of the species from the column occurred very close together in time. The separation of the four molecular species of the sample using a mobile phase comprising ammonium acetate were surprising nonetheless, given that ammonium acetate is known as anionic ion pairing agent, and anionic ion pairing agents are not expected to improve separation of a negatively charged oligonucleotide.

A reverse phase (i.e. hydrophobic) stationary phase and an ammonium acetate mobile phase were selected for further studies.

### Study 3D

In Study 3D, the conditions for Study 2 were carried out except that the gradient elution was carried out with decreasing concentrations of 100 mM ammonium acetate (MP A) and increasing concentrations of ACN (MP B). The details of the gradient mobile phase are as set forth in Table 2. The column flow rate was set at 0.5 ml/min; UV monitor at 260 nm, column temperature 35° C.

The results of this study are shown in FIG. 2H. As shown in this figure, all four molecular species of olpasiran (duplex, sense strand, antisense strand, and quadruplex) have differentiated retention times indicating that the method can separate all four molecular species if present in the same sample. Thus, the ammonium acetate gradient on the RP-HPLC C4 column was selected for further studies.

### Study 3E

In this study, the conditions for Study 3D were carried out with 100 mM ammonium acetate as MP A and ACN as MP B except that the gradient was slightly modified and the column flow rate was set at 0.8 ml/min. The details of the gradient were: 7% to 12% MP B in 5 min→12% to 14% MP B in 3 min→14% to 30% MP B in 7 min→30% MP B in 1 min→30% to 7% MP B in 2 min→7% MP B for 8 min.

The results of this study are shown in FIG. 2I. As shown in this figure, the resolution for the sense and antisense strands is improved and consistent with results of Study 3D, the method is able to separate all four molecular species, namely duplex, sense strand, antisense strand, and quadruplex.

### Study 4

In this study, the effect of column temperature of the Waters Xbridge BEH C4 column (2.1×50 mm, 300 Å, 3.5 μM) on the separation of the different molecular species of olpasiran was evaluated. After a sample comprising duplex, sense strand, and/or antisense strand of olpasiran was applied to the column, gradient elution was carried out with decreasing concentrations of 100 mM ammonium acetate (pH 7) (MP A) and increasing concentrations of ACN (MP B). The eluant was monitored at 260 nm and the column flow rate was 0.8 ml/min. The details of the gradient mobile phase are set forth in Table 4. The column temperature was 25° C., 30° C., 35° C., or 40° C.

FIGS. 2J and 2K provide exemplary chromatograms at each of the tested column temperatures. FIG. 2J shows the effect of temperature on the separation of the duplex (first peak in the chromatograms) and the sense strand (second peak in the chromatograms). FIG. 2K shows the effect of temperature on the separation of the antisense strand (first peak in the chromatograms) and quadruplex (G Quad, second peak in the chromatograms). Table 5 provides the area under the curve of each peak in FIG. 2K. Based on these results, the column temperature of 30° C. was selected as the optimal temperature.

TABLE 5

| Temp (° C.) | Antisense | G Quad |
|---|---|---|
| 25 | 5591729 | 11438801 |
| 30 | 5360386 | 11361759 |
| 35 | 5319552 | 11454086 |
| 40 | 5339497 | 11516898 |
| Average | 5402791 | 11442886 |
| StDev | 127057.2 | 63774.32 |
| % RSD | 2.35 | 0.56 |

One study was carried out at 50 degrees C. at a slightly modified gradient. It was found that this higher temperature moved the peaks corresponding to the single sense and antisense strands closer together providing a poorer separation of these two species.

### Study 5

In Study 1, a column comprising a chromatographic matrix comprising a C18 ligand was used, while in Studies 2, 3A-3C, 3D, 3E and 4, the chromatographic matrix comprised a C4 matrix. To evaluate the impact of the hydrophobic ligand of the chromatographic matrix on the separation of the different molecular species of olpasiran, a chromatographic matrix comprising a C3 ligand was used. IP—RP-HPLC was carried out using a Waters C3 column (2.1 mm×50 mm, 300 Å, 3.5 μm) maintained at 30° C. After a sample comprising the olpasiran duplex, sense strand, or antisense strand was applied to the column, gradient elution was carried out with decreasing concentrations of 100 mM ammonium acetate (pH 7) (MP A) and increasing concentrations of ACN (MP B). The details of the gradient mobile phase are set forth in Table 4.

The integrity of the duplex was lost with the C3 column as the duplex was resolved into separate phosphorothioate diastereomers. In addition, there was only about a 1-minute difference between the retention times for the sense and antisense strands. Thus, the C3 column did not improve resolution or separation of the molecular species.

### Study 6

In Study 2, a Waters Xbridge BEH C4 column (2.1×50 mm, 300 Å, 3.5 μM) was used. To evaluate the impact of the column length, a Waters Xbridge BEH C4 column with a longer column length (100 mm) was used. All other aspects of the column were the same as the column in Study 2. After a solution comprising olpasiran duplex, sense strand, or antisense strand (˜1 mg/mL) was injected into a Waters Xbridge BEH C4 column (2.1 mm×100 mm, 300 Å. 3.5 μm). A linear stepwise gradient elution was carried out with decreasing concentrations of 100 mM ammonium acetate (pH 7) (MP A) and increasing concentrations of ACN (MP B). The eluant was monitored at 260 nm; column temperature was 30° C. The column flow rate was 0.8 ml/min. Table 4 provides details of the mobile phase for gradient elution.

Exemplary results are shown in FIG. 2L. As shown in this figure, there was too much resolution for the duplex as the duplex began to separate into its phosphorothioate diastereomers. FIG. 2M provides exemplary results when the shorter column (Waters Xbridge BEH C4 column (2.1×50 mm, 300 Å, 3.5 μM) was used under nearly identical conditions. MP A was 100 mM ammonium acetate (pH 7) and MP B was ACN and the gradient parameters are provided in Table 4. Column temperature was 30° C. and column flow rate was 0.8 m/min. As shown in this figure, the duplex eluted as the peak at about 4.6 min (middle and bottom panels), the quadruplex eluted at about 12.9 min (top and middle panels), the sense strand eluted at about 6.2 min (bottom panel), and the antisense strand eluted at about 10.7 min (top panel). Although the resolution for the separation between the duplex and the sense strand (bottom panel) could be improved, FIG. 2M shows that this method can separate all four molecular species of olpasiran.

### Example 2

This example demonstrates the linearity for the response of the duplex when separated using the method described in Study 7 in Example 1 above with the Waters Xbridge BEH C4 column (2.1×50 mm, 300 Å, 3.5 μM), 100 mM ammonium acetate (pH 7)/ACN mobile phase and the gradient parameters provided in Table 4.

Linearity for the response of the duplex was assessed through serial dilutions of the olpasiran siRNA solution under identical conditions. An HPLC standardization curve for the duplex was prepared as follows: A series of standard solutions containing the olpasiran duplex at a concentration within the range of 0.01 mg/mL to 0.0875 mg/mL were prepared. These concentrations were determined by UV spectroscopy using 19.09 mL/mg*cm as extinction coefficient.

Standardization was achieved by measuring the HPLC peak areas of the solutions with known concentration (5 μL injection of sample). For each sample, the Waters Xbridge BEH C4 column (2.1×50 mm, 300 Å, 3.5 μm) was washed with a linear stepwise gradient system of 100 mM aqueous ammonium acetate (pH 7.0) containing increasing concentrations of CH₃CN in 100 mM aqueous ammonium acetate (7% to 12% MP B in 5 min, 12% to 14% MP B in 3 min, 14% to 30% MP B for 7 min, 30% for 1 min, 30% to 7% in 2 min, and back to baseline at 7% for 5 min at a flow rate of 0.8 mL/min. The eluant was monitored at 260 nm and the column temperature was 30° C.

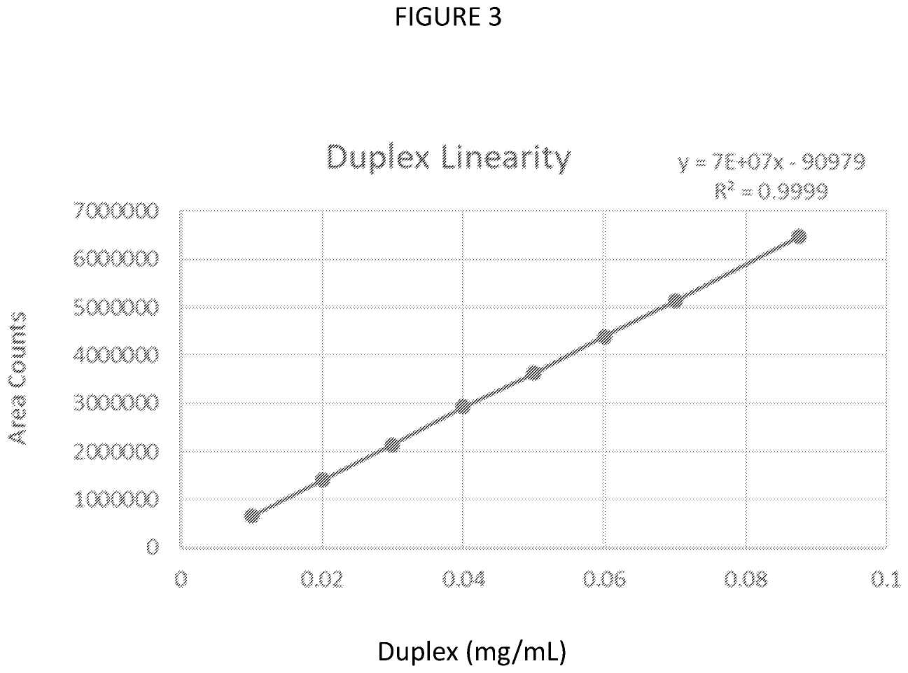

Under these conditions, the duplex eluted at 4.7 min. The molar extinction coefficient for the duplex at 260 nm was 15439 L cm-1 M-1. The long wavelength molar extinction coefficient was assessed for the purpose of evaluating the quadruplex. The peak areas versus concentrations were plotted, the “R-squared value” was 0.999. Linearity is shown graphically in FIG. 3.

This example demonstrated excellent linear correlation between UV 260 nm response of duplex peak to the duplex concentration within a range from 0.01 mg/mL to 0.08 mg/mL.

### Example 3

This example describes the impact of solution preparation on the antisense strand::quadruplex ratio.

In a study aimed at analyzing the impact of the solution in which the olpasiran sample is prepared on the antisense strand::quadruplex ratio (which allows insight as to the equilibrium between the antisense strand and quadruplex), solutions containing the sense strand (A10B), antisense (strand (A10A), or duplex (A10C) were prepared in a solvent described in Table 6. The solutions were stored at room temperature for 2 h, then placed at 5° C. in the autosampler for injection. The column was washed with a linear stepwise gradient system of 100 mM aqueous ammonium acetate (pH 7.0) containing increasing concentrations of ACN in 100 mM aqueous ammonium acetate and the gradient parameters are provided in Table 4. The flow rate of 0.8 mL/min. The eluant was monitored at 260 nm and the column temperature was 30° C.

TABLE 6

| | Solvent | Shorthand |
|---|---|---|
| Sense | 9.22 mg- | A10B-N |
| (A10B) | dissolved in 1 mL NH₄OAc (100 mM) | |
| | 8.30 mg-dissolved in 1 mL Water (wifi) | A10B-W |
| | 7.11 mg-dissolved in 1 mL HFIP/TEA | A10B-H |
| Antisense | 17.89 mg-dissolved in 1 mL | A10A-N |
| (A10A) | NH₄OAc (100 mM) | |
| | 21.30 mg-dissolved in 1 mL Water (wifi) | A10A-W |
| | 18.73 mg-dissolved in 1 mL HFIP/TEA | A10A-H |
| Duplex | 19.52 mg-dissolved in 1 mL | A10C-N |
| (A10C) | NH₄OAc (100 mM) | |
| | 13.50 mg-dissolved in 1 mL Water (wifi) | A10C-W |
| | 20.31 mg-dissolved in 1 mL HFIP/TEA | A10C-H |

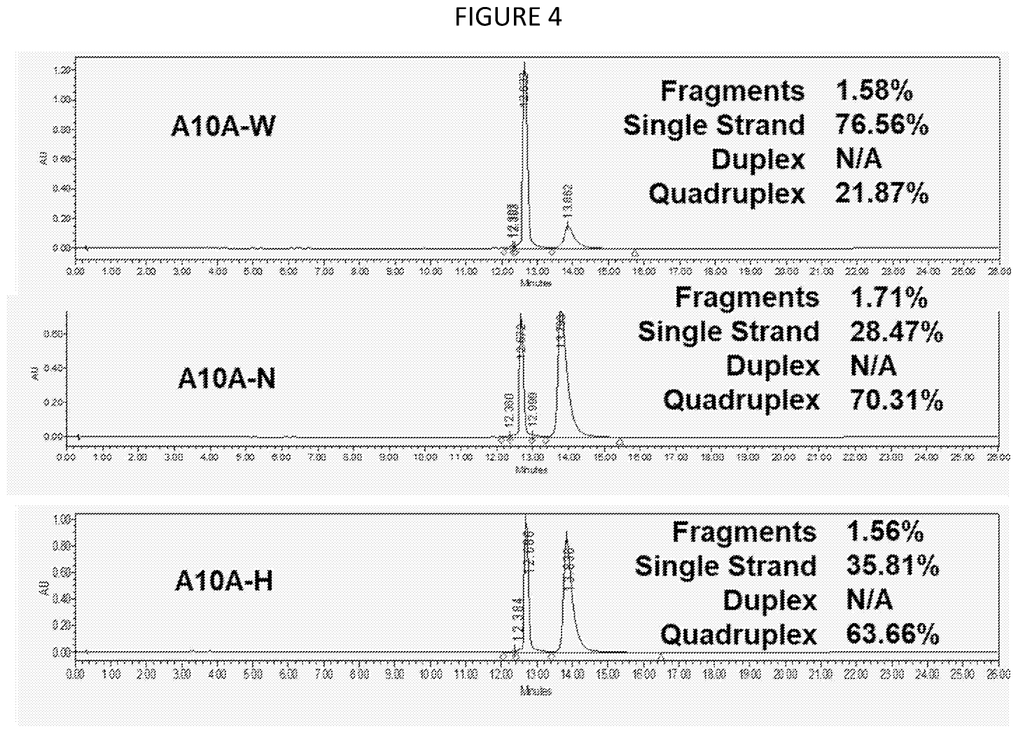

Exemplary chromatograms of the antisense samples are provided in FIG. 4. As shown in the top chromatogram (A10A-W) of FIG. 4, the amount of the early eluting peak, the antisense strand, was noticeably higher than the later peak, the quadruplex (76.56% vs. 21.87%). Thus, water does not appear to support the quadruplex structure. As shown in the middle and bottom chromatograms of FIG. 4, the two samples of antisense strand that were prepared in HFIP/TEA (bottom chromatogram) or ammonium acetate (middle chromatogram) were supportive of the tetrad (quadruplex) structure as based on peak integration. Samples of antisense strand prepared in ammonium acetate led to a higher amount of quadruplex (70.31%) compared to water (21.87%). Samples of antisense strand prepared HFIP/TEA also led to a higher amount of quadruplex (63.66%) compared to water (21.87%), but not as high as ammonium acetate (70.31%).

A separate study was carried out to analyze the effects of sample preparation on the quadruplex. Solutions containing the antisense strand (A10A) were prepared in either 1) water or 2) ammonium acetate (100 mM) as detailed in Table 7.

TABLE 7

| | Actual | |
|---|---|---|
| | Concentration | |
| | by UV | |
| Concentration | (27.95 | Solvent |
| of A10A | mL/mg*cm) | (volume) |
| 55.6 mg per 2 mL | 23.33938 mg/mL | Water (2 mL) |
| 64.6 mg per 3 mL | 17.4363 mg/ml | 100 mM |
| | | NH₄OAc (3 mL) |

The higher concentrations of the undiluted solutions caused a bending of the Absorbance/Pathlength curve, so the samples were diluted by 10. The diluted concentrations were used for results. A solution containing 100 μL of each solution was heated at 65° C. for 20 min then cooled to RT. The control was not heated. Solutions were diluted 10-fold and loaded in cuvette for SoloVPE analysis.

After heating, aliquots were pulled and diluted 10× for Conc Determination:

$$Antisense in {NH}_{4} OAc - Post - Heat conc by UV ( 27.95 mL / mg * cm ) = 19.093 mg / mL ( 9.5 \% increase )$$

$$Antisense in Water - Post - Heat conc by UV ( 27.95 mL / mg * cm ) = 24.8888 mg / mL ( 6.64 \% increase )$$

Purity analysis was conducted using the separation method described in Study 6 in Example 1 with the Waters Xbridge BEH C4 column (2.1×50 mm, 300 Å, 3.5 μM), 100 mM ammonium acetate (pH 7)/ACN mobile phase and the gradient parameters provided in Table 4 except the column temperature was adjusted to 8° C.

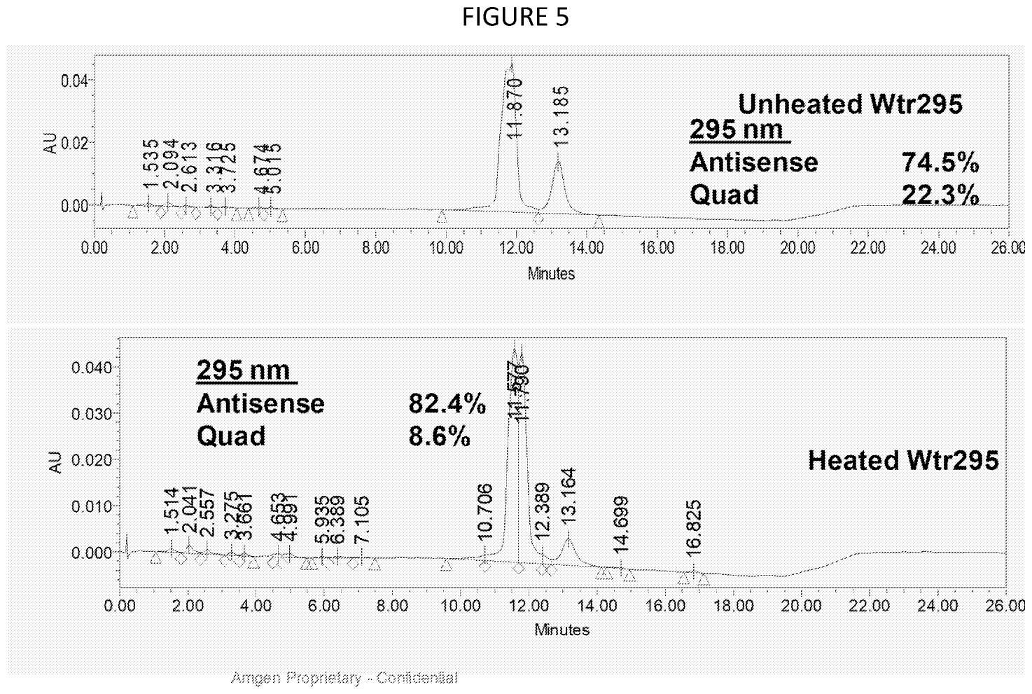

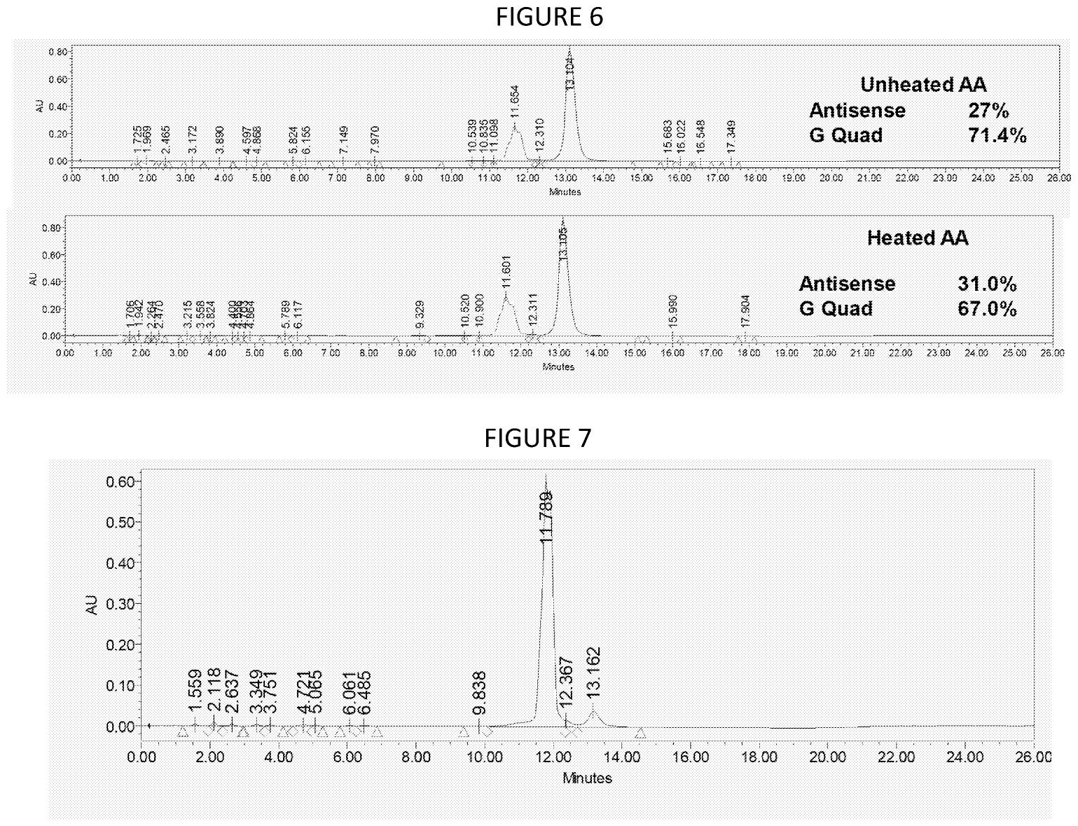

The results are shown in FIGS. 5 and 6 and Table 8.

TABLE 8

| Solvent | Heat Treatment | % Antisense | % Quadruplex |
|---|---|---|---|
| Water | + | 82.4 | 8.6 |
| Water | − | 74.5 | 22.3 |
| NH₄OAc | + | 31 | 67 |
| NH₄OAc | − | 27 | 71.4 |

The heated sample using water as the dissolution medium showed a very different profile as compared to ammonium acetate. When the sample was prepared in water, the heat disrupted the quadruplex, shifting the equilibrium to the antisense strand. The early eluting peak increased significantly after heating, which indicated that the early peak was the monomer, antisense strand. Heat also disrupted the quadruplex in samples prepared in ammonium acetate, but the shift in the equilibrium from quadruplex to antisense strand was significantly reduced, suggesting that the ammonium ion partially stabilizes the quadruplex.

Taken together, these results demonstrate that the detectable amounts of antisense and quadruplex can vary depending on the solution preparation solution. In some cases, it is beneficial to prepare the samples in a solution containing an ion that will stabilize the quadruplex, such as ammonium or potassium ions, such that the ratio of antisense strand to quadruplex will not shift during separation and the quantitation of each of these molecular species will be more accurate.

### Example 4

This example demonstrates the linearity of the response for the quadruplex when separated using the method described in Study 6 in Example 1 above with the Waters Xbridge BEH C4 column (2.1×50 mm, 300 Å, 3.5 μM), 100 mM ammonium acetate (pH 7)/ACN mobile phase and the gradient parameters provided in Table 4.

Linearity for the quadruplex was assessed using the heated A10A sample in water described in Example 3.

An HPLC standardization curve for the quadruplex was prepared by measuring the HPLC peak areas of the solutions with known concentration, as essentially described in Example 2. The column and gradient elution were as described in Example 2. The eluant was monitored at 260 nm and the column temperature was 8° C.

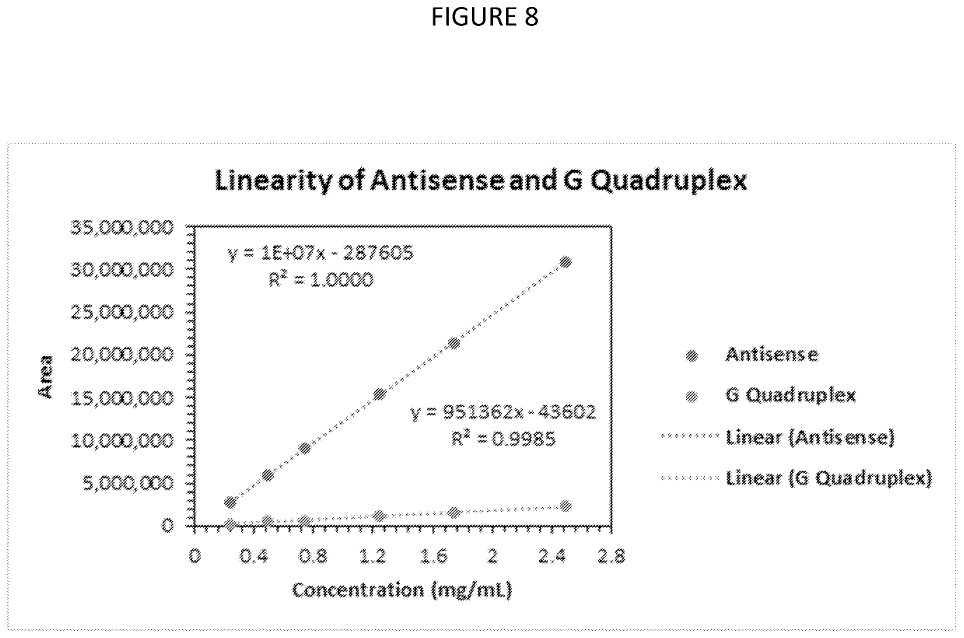

An exemplary chromatogram of the antisense/quadruplex equilibrium in heated samples comprising a water solvent is provided in FIG. 7. As shown in this figure, under these conditions, the antisense and quadruplex eluted at 11.8 min and 13.2 min, respectively. A reduction in the column temperature (8° C.) relative to the column temperature of Example 2 (30° C.) was used to stabilize both the antisense and G quadruplex peak shape. Since the extinction coefficient is unknown the concentration of the G quadruplex cannot be determined. The peak areas for each of the antisense strand and quadruplex versus concentrations of sample are plotted in the graph of FIG. 8. The “R-squared value” for both the antisense strand and the quadruplex was 1.0.

### Example 5

This example demonstrates the effect of potassium on stabilizing the quadruplex.

Samples comprising olpasiran antisense strand were prepared in a solution with or without 100 mM potassium and then were subjected to heat treatment. Controls were not subjected to the heat treatment. The samples were applied to a Waters Xbridge BEH C4 column (2.1×50 mm, 300 Å, 3.5 μM) and the gradient elution was carried out with decreasing concentrations of 100 mM ammonium acetate (pH 7) (MP A) and increasing concentrations of ACN (MP B). The details of the gradient mobile phase are set forth in Table 9. The column flow rate was set at 0.8 ml/min. The elution was monitored using a UV monitor at 260 nm. The column temperature was 8° C.

TABLE 9

| Time | % MP A | % MP B |
|---|---|---|
| 0 | 93 | 7 |
| 5 | 88 | 12 |
| 8 | 88 | 12 |
| 11 | 86 | 14 |
| 18 | 70 | 30 |
| 19 | 70 | 30 |
| 21 | 93 | 7 |
| 26 | 93 | 7 |

Potassium appears to drive the equilibrium between the antisense strand and quadruplex towards the quadruplex and stabilizes the quadruplex even when the quadruplex is subjected to heat treatment as the peak area for the quadruplex increases relative to the peak area for the antisense strand in the presence of potassium. In the absence of potassium, the heat treatment destroys the quadruplex and the structure reverts to antisense single strands as evidenced by the significantly reduced peak corresponding to the quadruplex and increase in peak area for the peak corresponding to the antisense strand. Even without potassium, the quadruplex is stable enough for detection by this method. It is believed that the ammonium ion in the mobile phase acts to stabilize the quadruplex.

This example supports the use of potassium in sample preparation to stabilize the quadruplex structure and prevent shifts in the ratio of antisense strand to quadruplex during separation.

### Example 6

This example demonstrates an exemplary method of separating the molecular species of a G-rich oligonucleotide.

In a first method, RP-HPLC was carried out using a Waters Xbridge BEH C4 column (2.1×50 mm, 300 Å, 3.5 μM). The column temperature was 30° C. Samples comprising olpasiran duplex, antisense strand, sense strand, or G-quadruplex (formed from olpasiran antisense strands) were prepared in deionized water. Specifically, duplex sample solution was prepared at ˜70 mg from lyophilized power dissolved with 1 mL deionized water into a polypropylene vial. Both sense strand and antisense strand sample solutions were provided at ˜30 mg/mL in water which were diluted to ˜4.5 mg/mL with deionized water. Enriched G-Quadruplex solution (>96% area) obtained by incubating the antisense strand with various cations in a 3:5 ratio at room temperature for up to 1 week was provided at ˜3.5 mg/mL in sodium phosphate with acetonitrile and NaBr (625 mM final concentration) buffer and was directly analyzed without further dilution.

After these prepared olpasiran samples were injected into the autosampler, gradient elution was carried out with decreasing concentrations of 100 mM ammonium acetate in water (pH 6.8) (MP A) and increasing concentrations of ACN (MP B). The details of the gradient mobile phase are set forth in Table 9 above. The column flow rate was set at 0.8 ml/min. The elution was monitored using a UV monitor at 260 nm/4 nm bandwidth. The total run time was 26 minutes.

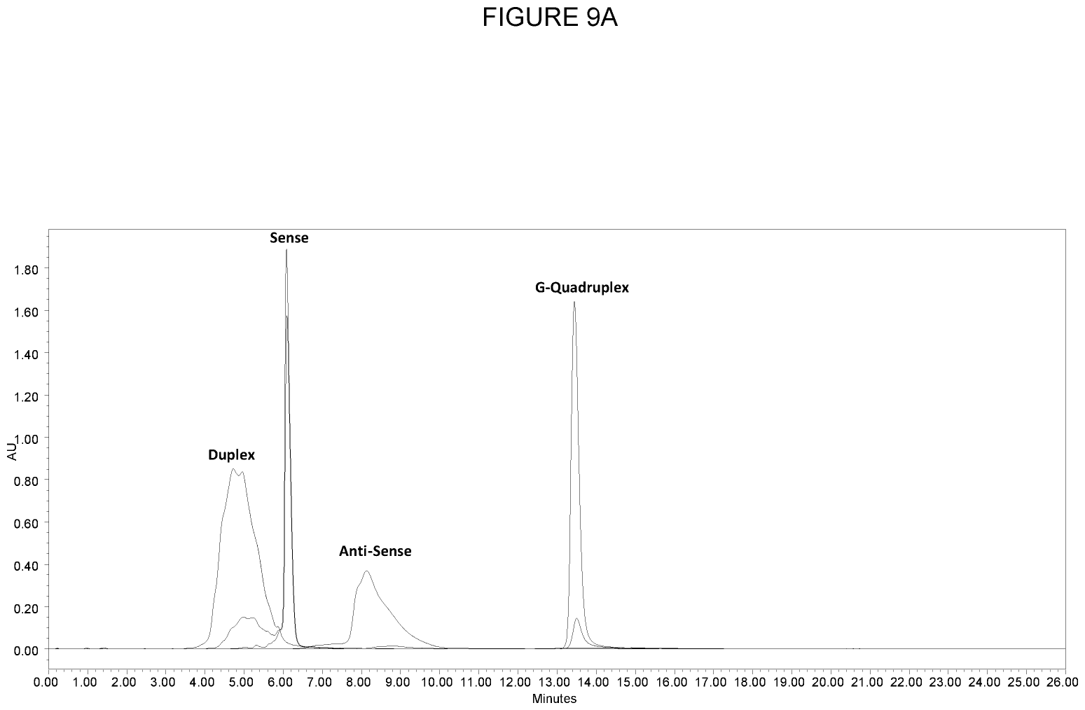

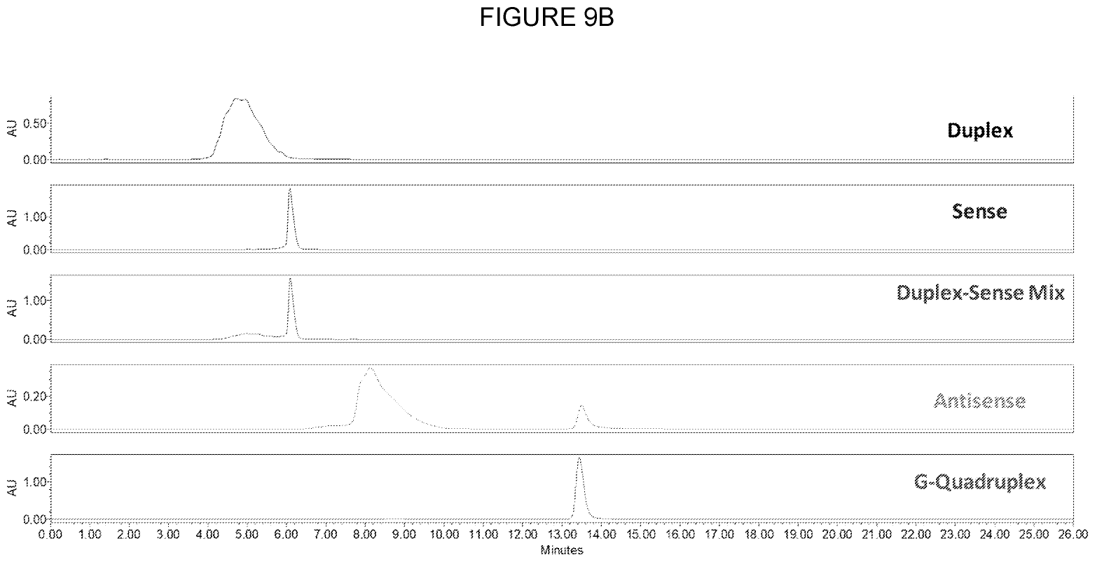

FIGS. 9A and 9B depict the chromatograms of the molecular species as overlay and stacked views, respectively. As shown in these figures, all four molecular species can be detected by the method. However, resolution between the duplex peak and sense strand peak (USP resolution 51.2) could be improved.

To improve the resolution of the duplex peak and sense strand peak, a second RP-HPLC method using the same C4 column similar to the first method was carried out. The second method (“Method 2”) was identical to the first method except that the mobile phase of the second method comprised a decreasing concentration of 75 mM ammonium acetate in water (pH 6.8)(MP A) and increasing concentration of ACN (MP B) according to different gradient parameters as set forth in Table 10. The flow rate also was decreased to 0.7 m/min and the total run time was 30 minutes. The autosampler temperature was 15 degrees C.

TABLE 10

| Time (min) | MP A (%) | MP B (%) |
|---|---|---|
| 0 | 92 | 8 |
| 2 | 90 | 10 |
| 18 | 86 | 14 |
| 26 | 70 | 30 |
| 26.1 | 92 | 8 |
| 30 | 92 | 8 |

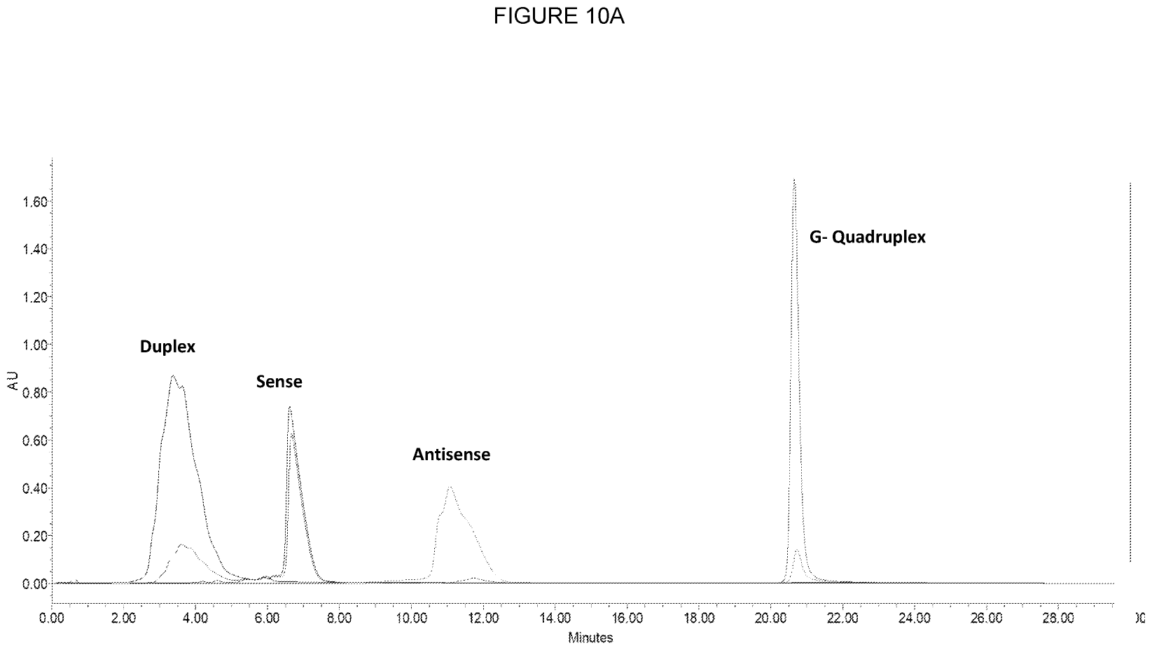

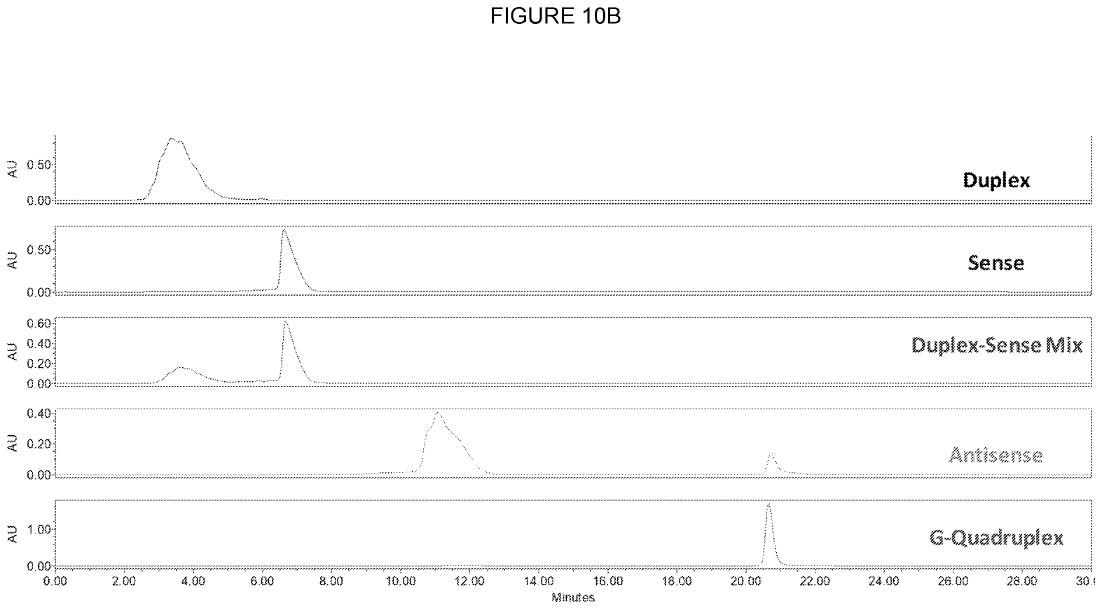

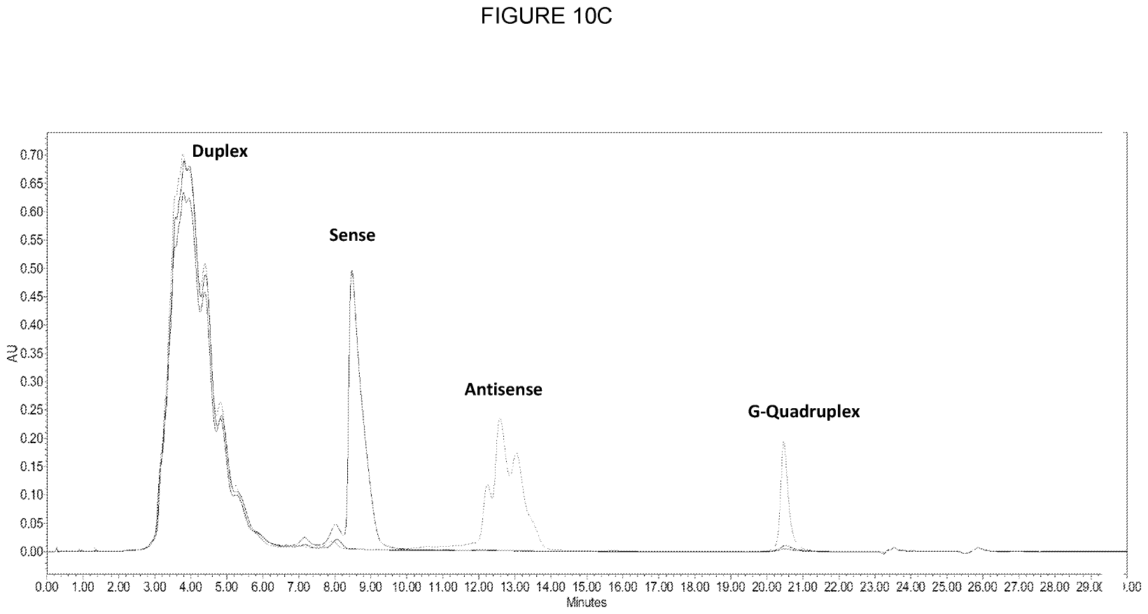

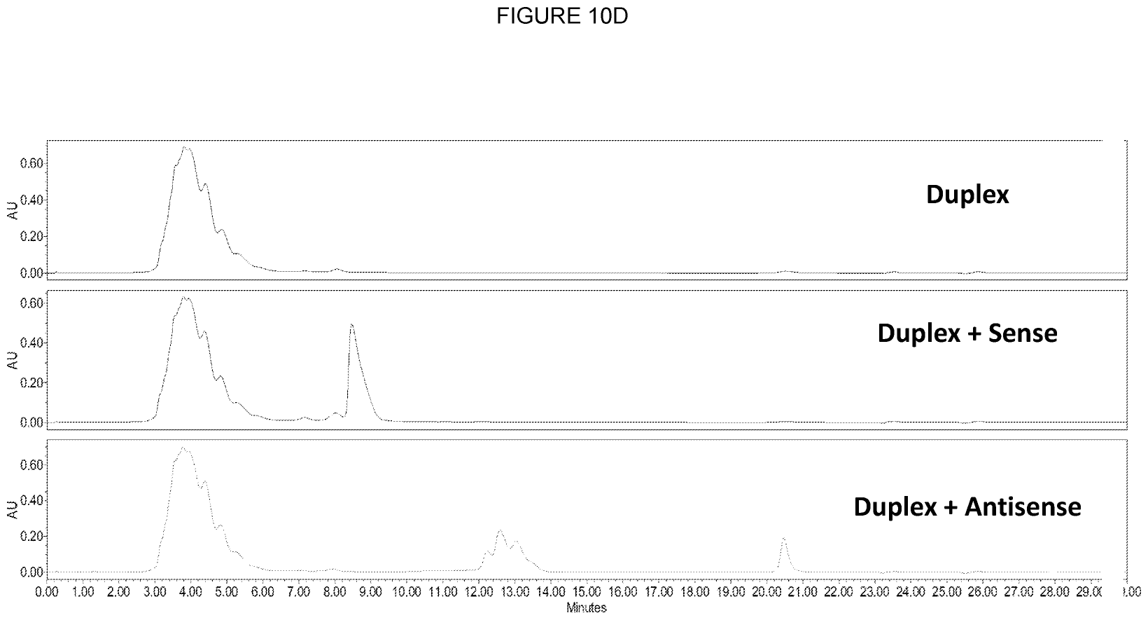

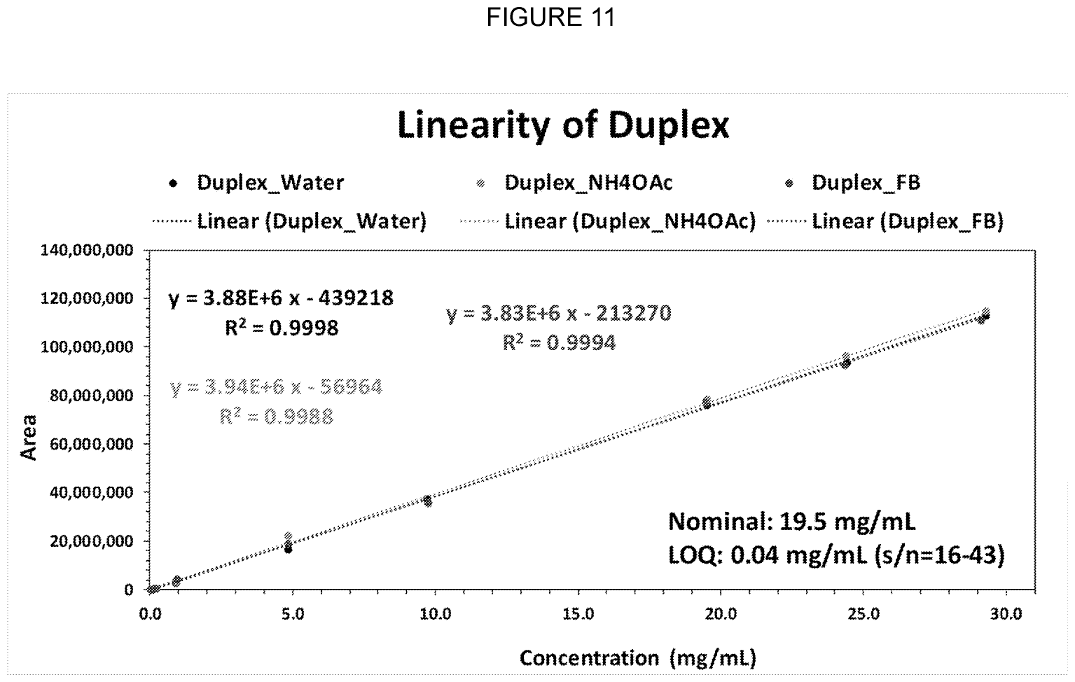

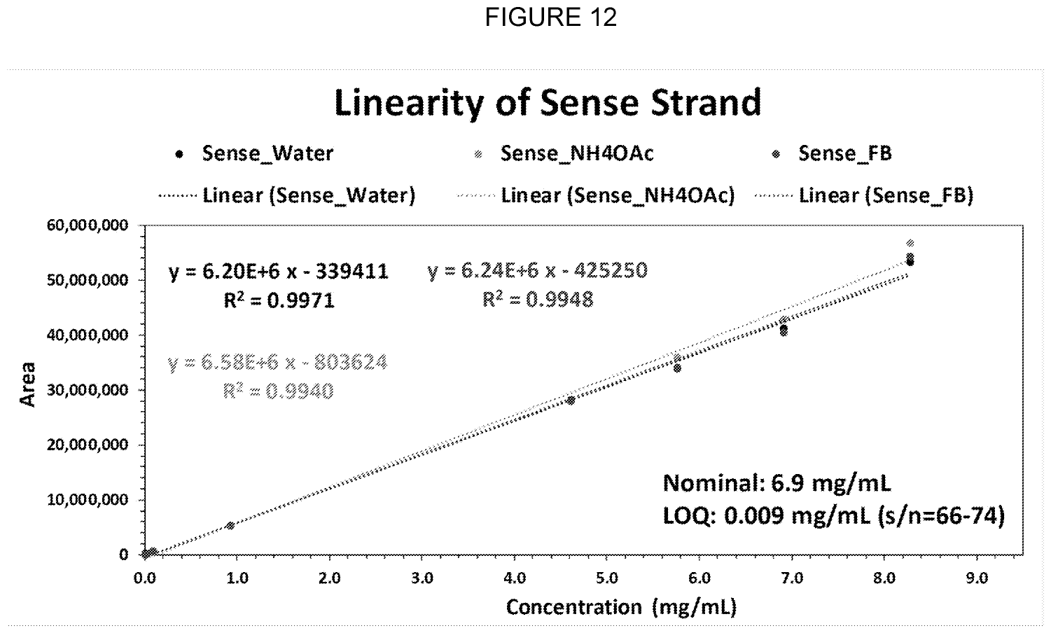

FIGS. 10A and 10B depict the chromatograms of the molecular species as overlay and stacked views, respectively. As shown in these figures, the duplex and sense strand peaks separated well from each other (USP resolution 22.4). Also, this method improved the separation between the sense strand and antisense strand peaks as well as separation between the antisense strand peak and quadruplex peak.

To avoid potential carryover issues, a third RP-HPLC method using the same C4 column similar to the first and second methods was carried out. The third method (“Method 3”) was identical to the second method wherein a Waters XBridge Protein BEH C4 column (2.1 mm×50 mm, 300 Å, 3.5 μm), except that an additional column flushing step was added after the quadruplex elution was completed. The additional flushing step occurred from 22.1 min to 24 min. The details of the mobile phase gradient parameters are set forth in Table 11. Also, for the acetate gradient, a stock solution of 75 mM ammonium acetate in water (pH 6.7±0.1) was used. The flow rate was 0.7 ml/min±0.2 m/min, and the total run time was 30 minutes. The autosampler temperature was 15° C.±1° C. The column temperature was 30° C.±1° C. Elution was monitored by UV at 260 nm (4 nm bandwidth for Agilent LC system or 4.8 nm bandwidth for Waters UPLC system).

TABLE 11

| Time (min) | Acetate (%) | Acetonitrile (%) |
|---|---|---|
| 0.0 | 92 | 8 |
| 2.0 | 90 | 10 |
| 18.0 | 86 | 14 |
| 22.0 | 78 | 22 |
| 22.1 | 20 | 80 |
| 24.0 | 20 | 80 |
| 24.1 | 92 | 8 |
| 30.0 | 92 | 8 |

Samples were prepared in purified, deionized water. Acetate stock solution for gradient was 75 mM ammonium acetate in water, pH 6.7 ± 0.1. Acetonitrile stock solution was 100% acetonitrile.

The results are shown in FIGS. 10C and 10D. As shown in this method, the details of Method 3 did not change the elution profiles of the peaks of the molecular species observed with Method 2. This was expected given that the gradient steps before the column flushing step remained the same. All four molecular species were chromatographically separated with high resolution.

### Example 7

This example describes a study to evaluate different sample diluents.

Sample solutions were prepared in three different sample diluents, (1) deionized water, (2) 75 mM ammonium acetate in water at pH 6.8, and (3) a drug product formulation buffer (20 mM potassium phosphate with 40 mM sodium chloride in water at pH 6.8). Samples were then separated using Method 2 described in Example 6 above. All results were compared to evaluate the method linearity and any effect with different sample diluents.

First, a nominal concentration (100% level) for each of the molecular species (antisense strand, sense strand, duplex, quadruplex) was determined at the concentration which gave its main peak height at ˜1.0 AU (absorbance unit). Second, after a series of dilutions, the lowest concentration was determined as the limit of quantification (LOQ) level for each main peak which gave the signal-to-noise (s/n) value of the peak greater than 10.0. The sample concentration ranges covering from LOQ to 120% of the nominal concentration were selected to evaluate the method linearity for each molecular species.

FIG. 11 shows the linearity response of duplex peak area versus its concentration covering from LOQ to 150% of the nominal concentration which were prepared in three different diluents. Duplex samples in both water and formulation buffer (FB) showed no difference and gave same highly linear responses with R² values of 0.9998 and 0.9994, respectively. Duplex samples in 75 mM ammonium acetate also gave a highly linear response with a R² value of 0.9988. The nominal concentration of duplex was determined at 19.5 mg/mL and LOQ level at 0.04 mg/mL (0.20% of nominal concentration). Sample testing and method qualification was also successfully completed by using a decreased nominal concentration of the duplex (15 mg/mL). In this instance, a very high linearity response with the R² of 0.9993 of duplex peak area versus its concentration was achieved. The LOQ level was 0.08 mg/mL and the signal-to-noise ratio was 26-28.

The stock solutions of sense strand and antisense strand at ˜30 mg/mL were used to prepare a series of diluted sample solution for the linearity evaluation of these single strands and G-Quadruplex. Accurate concentration measurement of these stock solutions was performed by Solo VPE by using their extinction coefficients at 260 nm, 21.74 mL/mg*cm (sense) and 27.93 mL/mg*cm (antisense). The concentrations of the stock solutions measured were 27.63 mg/mL for sense strand and 32.78 mg/mL for antisense strand. The concentration ranges covering from LOQ to 120% of nominal concentration were selected for sense strand, antisense strand, and G-Quadruplex. All showed highly linear responses of peak area versus concentration with R² values greater than 0.99 as shown in FIG. 12, FIG. 13, and FIG. 14, for the sense strand, antisense strand and quadruplex, respectively.

The nominal concentration of sense strand was determined at 6.9 mg/mL, and LOQ at 0.009 mg/mL (0.13% of the nominal). The linearity evaluation of antisense and G-Quadruplex was performed at the same time with the same samples because all antisense strand samples also contained G-Quadruplex at ˜19% (area %). The nominal concentration and LOQ of the antisense strand were at 13.3 mg/mL and 0.005 mg/mL (0.038% of nominal), respectively. And the nominal concentration and LOQ of G-Quadruplex were at 3.0 mg/mL and 0.03 mg/mL (1% of nominal), respectively.

For each molecular species, there was no significant difference among the tested sample diluents when run under these conditions. Samples in water and DP formulation buffer showed exactly same responses in their linearity evaluations. These results support the use of deionized water (Resistivity ≥18 Ω cm) as the sample diluent in instances wherein, for example, driving the equilibrium between antisense and quadruplex to quadruplex is not desired.

### Example 8

This example describes the impact of heating—cooling treatment on the antisense and quadruplex.

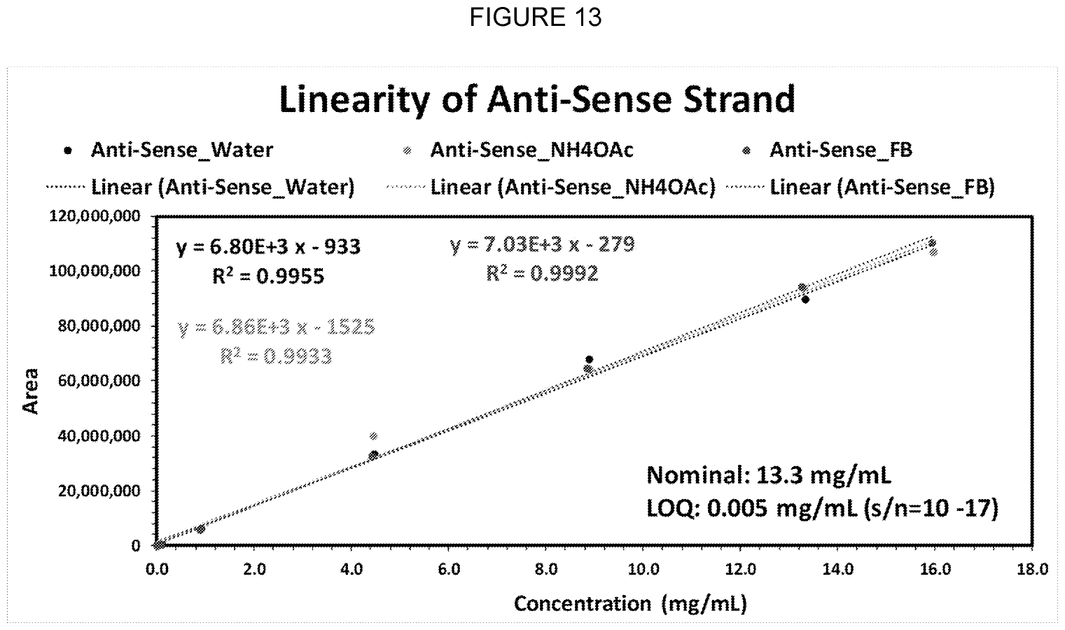

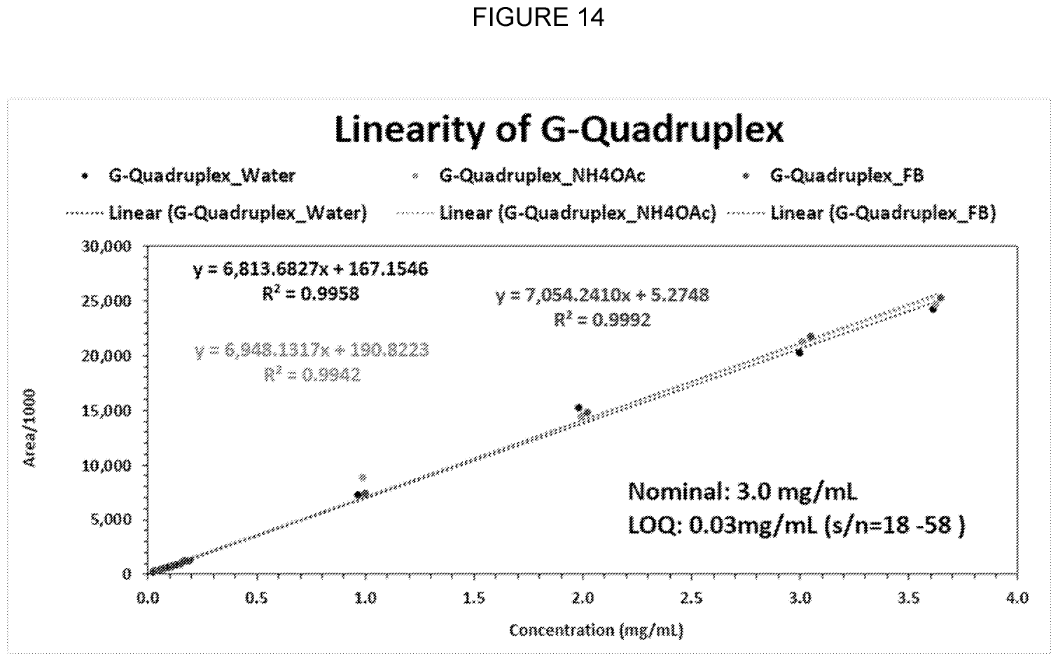

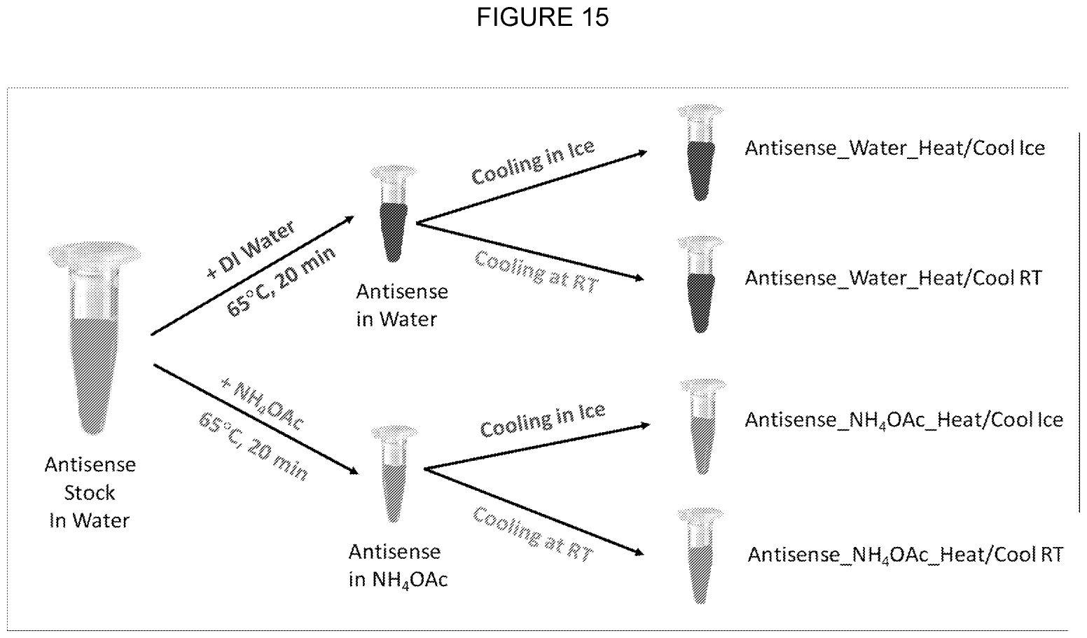

Solutions comprising olpasiran antisense strands (8.2 mg/mL) were prepared by diluting antisense stock solution with one of two different diluents: deionized water and 75 mM ammonium acetate in water at pH 6.8. The diluted antisense strand solutions were exposed to heat at 65° C. for 20 minutes. After the heat treatment, each solution was cooled down on ice or at room temperature (RT). FIG. 15 depicts the sample preparation procedure.

Solutions were analyzed by Method 2 described in Example 6 to evaluate the effect of the diluent and of the heating-cooling treatment. In particular, the % area of antisense peak and G-Quadruplex peak for each sample was measured.

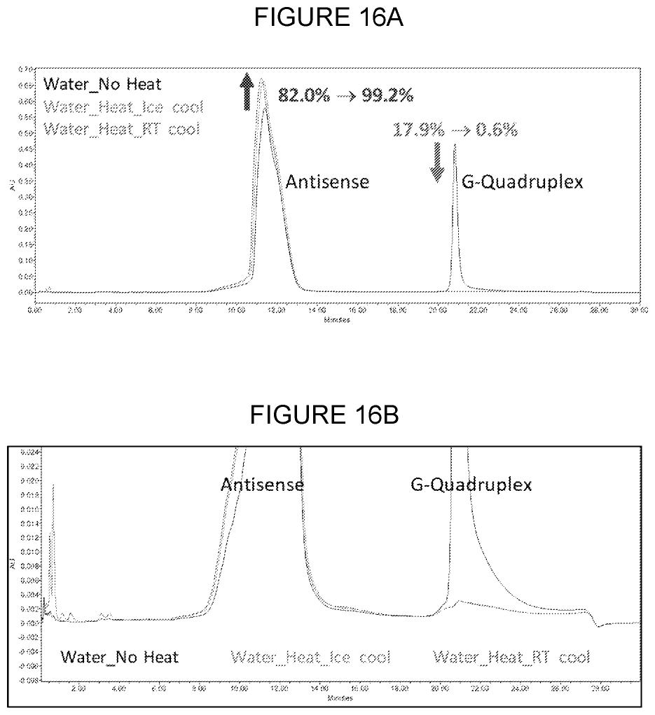

FIGS. 16A and 16B show the overlay chromatograms of the antisense strand solutions prepared in water before and after the heating-cooling treatment. The % area of the antisense peaks dramatically increased from 82.0% to 99.2% after the heating-cooling treatment, compared to the sample without heat-treatment. This increase (17.2%-increase) in antisense strand content correlates with the decrease in the % area of the quadruplex (17.3%-decrease). There was no difference observed between the two different cooling processes (ice vs. RT).

FIGS. 17A and 17B show the overlay chromatograms of the antisense strand solutions in 75 mM ammonium acetate buffer before and after the heating-cooling treatment. Interestingly, there was no significant change observed in the % Area of the antisense peaks as well as G-Quadruplex peaks (e.g., before vs. after heat-treatment and cooling in ice vs. cooling at RT). The % Area of the Antisense peak and G-Quadruplex peak remained the same at ˜82% and ˜18%, respectively, in all solutions. This result clearly demonstrates a strong stabilizing effect of ammonium cation in NH₄OAc buffer on G-Quadruplex during the heating/cooling processes, compared to the heated samples in water. The heat-disrupted G-Quadruplex resulted in equilibrium shift more to the antisense strand (a monomer) in water. However, this heat disrupted, weakened G-Quadruplex structure seemed quickly stabilized by the ammonium cation in the ammonium acetate sample diluent and ultimately resulted in no significant changes in G-Quadruplex content in the final solutions in ammonium acetate buffer.

This example supports preparing samples of the G-rich oligonucleotide in ammonium acetate to stabilize the equilibrium between the G-rich oligonucleotide and the quadruplex.

### Example 9

This example demonstrates a cation effect of mobile phase buffers on the antisense::quadruplex equilibrium during HPLC.

In Example 8, the G-Quadruplex-stabilizing effect of the ammonium acetate as a sample diluent was clearly demonstrated. In this example, the effect of sodium acetate (NaOAc) and potassium acetate (KOAc) on the antisense::quadruplex equilibrium was evaluated.

Solutions comprising olpasiran antisense strands at a nominal concentration of 4.5 mg/mL or 13.3 mg/mL were prepared by diluting antisense stock solution with one of three different diluents: deionized water, 75 mM NaOAc in water at pH 6.8, or 75 mM KOAc in water at pH 6.8. Each solution was analyzed by a method similar to Method 2 described in Example 6, except that the mobile phase A solution comprised 75 mM NaOAC in water at pH 6.8 or 75 mM KOAc in water at pH 6.8. The % area for each of the antisense peak and the G-Quadruplex peak was measured.

The results are shown in Table 12.

TABLE 12

| Nominal Conc | | | % area of | % area of |
|---|---|---|---|---|
| of Antisense | Sample Prep | Mobile Phase | antisense | quadruplex |
| (mg/ml) | Diluent | Component | peak | peak |
| 4.5 | Water | NaOAc | 84.41 | 15.59 |
| | NaOAc | NaOAc | 83.22 | 16.78 |
| | KOAc | NaOAc | 82.71 | 17.29 |
| 4.5 | Water | KOAc | 84.09 | 15.91 |
| | NaOAc | KOAC | 82.87 | 17.13 |
| | KOAc | KOAC | 82.50 | 17.50 |
| 13.3 | Water | NaOAc | 82.57 | 17.43 |
| | NaOAc | NaOAc | 82.55 | 17.45 |
| | KOAc | NaOAc | 82.00 | 18.00 |
| 13.3 | Water | KOAc | 81.62 | 18.38 |
| | NaOAc | KOAc | 81.41 | 18.59 |
| | KOAc | KOAC | 81.21 | 18.79 |

For the 4.5 mg/mL antisense concentration, there was a decreasing trend in % area of the antisense peaks and a corresponding increasing trend in the % area of the quadruplex peaks among the different diluents with KOAc exhibiting the lowest % area of the antisense peak and the highest % area of the quadruplex peak. Moreover, KOAc mobile phase showed higher quadruplex content and lower antisense content than NaOAc mobile phase did. For samples with higher antisense concentration (13.3 mg·mL), similar decreasing antisense content with concurrently increasing quadruplex content were observed with larger changes in both decreasing antisense content and increasing quadruplex content. These results showed the antisense-quadruplex equilibrium further shifted to favor more quadruplex formation at the higher antisense concentration (13.3 mg/mL) than that at 4.5 mg/mL. As expected, KOAc showed the highest stabilizing effect among three different sample diluents and KOAc mobile phase is more favorable for quadruplex structure than NaOAc.

### Example 10

This example describes the characterization of G Quadruplex by other analytical techniques.

The G-quadruplex structure incorporates four of the G-rich antisense strand monomers with cations (NH₄⁺, Na⁺, or K⁺) held non-covalently between the strands. The formation of this structure would result in a mass increase from ˜7020 Da antisense monomer) to ˜28100 Da (G-quadruplex) as observed in Kazarian et al., Journal of Chromatography A. Vol 1634: 461633 (2020) for the same species. Several analytical techniques were used to provide further evidence that a G-quadruplex structure was detected using the RP-HPLC methods described in the previous Examples, including liquid chromatography-mass spectrometry (LC-MS) and dynamic light scattering (DLS). The results of these analytical tests are discussed below.

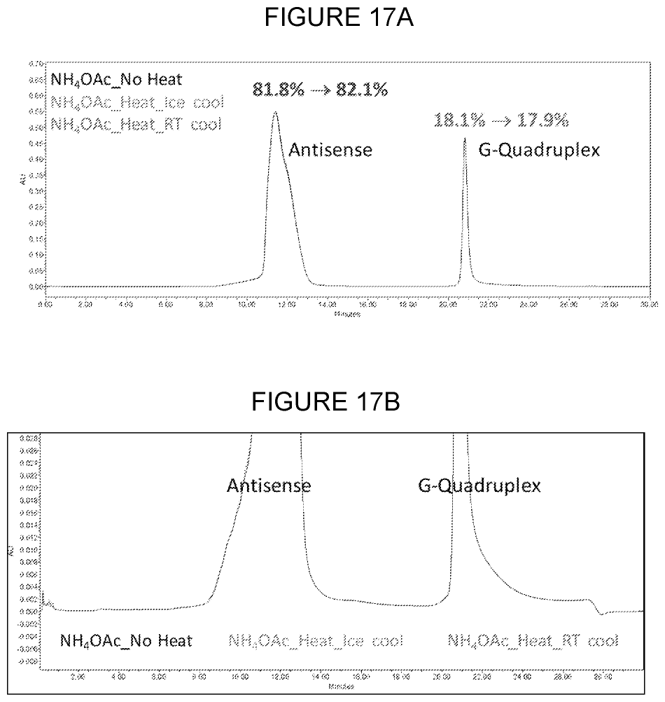

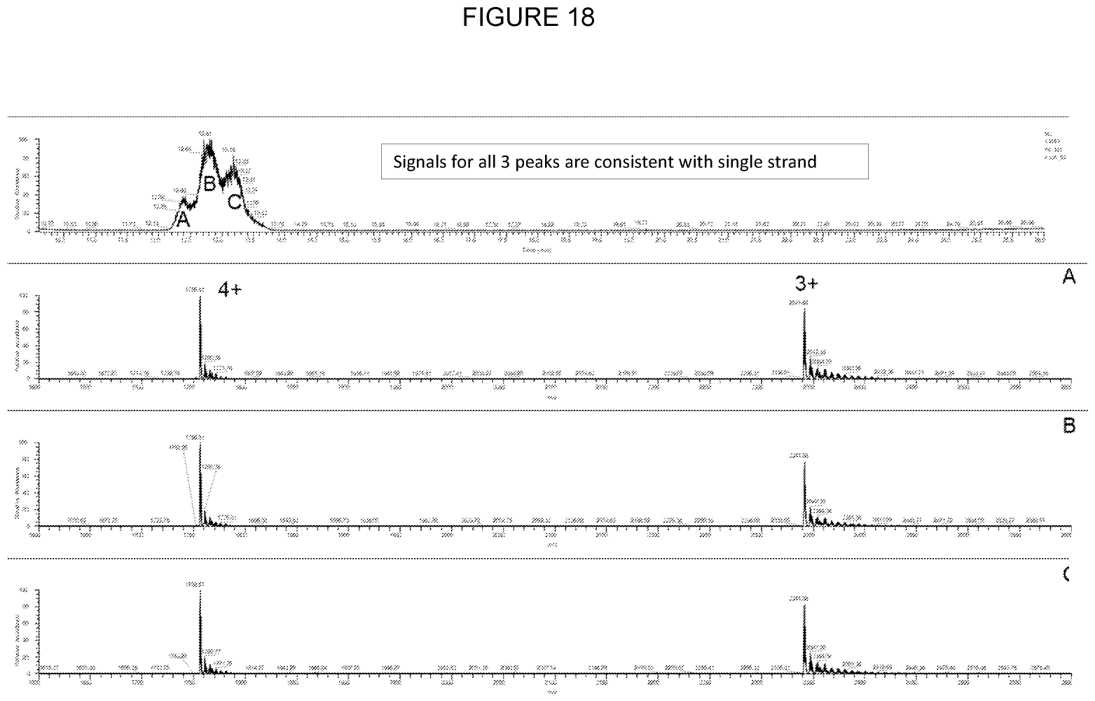

LC-MS: LC-MS analysis was performed of both the antisense strand and G-quadruplex samples. The G-quadruplex sample was obtained by incubating antisense strand with NaBr for 1 week. Data was collected using an Agilent 1290 Infinity II LC in line with a Thermo Scientific QExactive HFX mass spectrometer. Baseline separation of the two species was achieved on a column with the same C4 stationary phase but slightly different dimensions. The MS spectrum associated with the proposed antisense single strand provides a narrow charge state distribution of the 3+ and 4+ charge states (FIG. 18). The multiple peaks observed in the main proposed single strand peak are most likely due to phosphorothioate diastereomers resulting from differences in chirality introduced by the presence of phosphorothioate bonds in the sequence. In the antisense sample, no clear MS signal was observed for the proposed G-quadruplex peak that corresponded to the single strand or G-quadruplex.

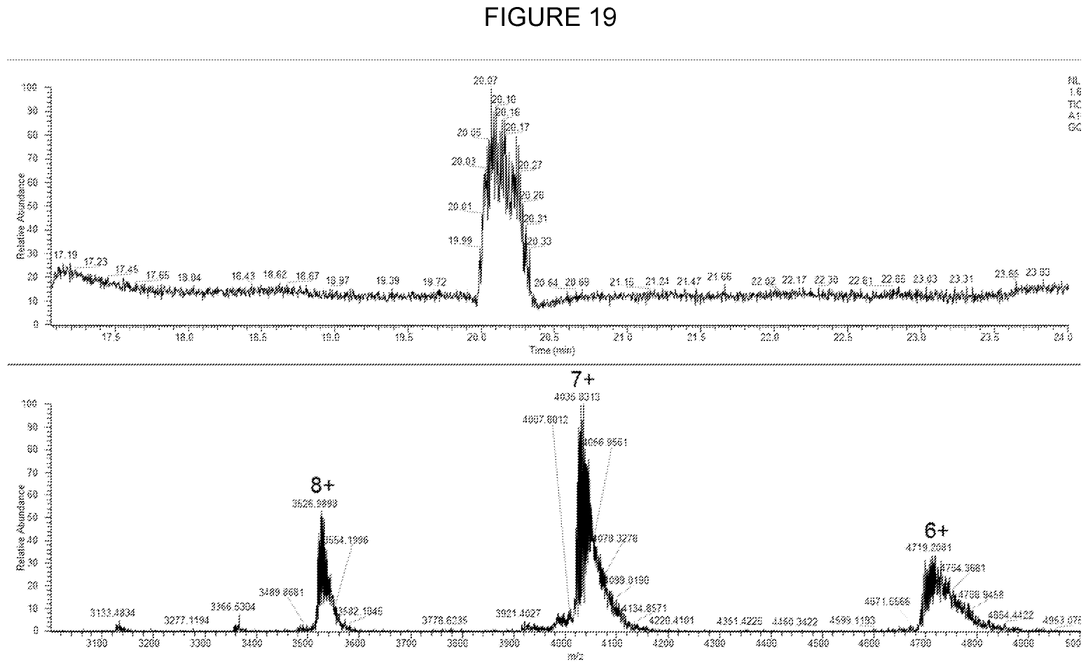

However, when an MS spectrum was pulled from the concentrated G-quadruplex sample, MS signals were observed at higher m/z (FIG. 19). These MS signals, though heavily adducted with various cations (Water, Na⁺, and NH₄⁺) do correspond to a larger structure and no single strand signal is observed. Additionally, no MS signal was observed at the m/z where single stranded antisense signal should be present. This observation supports the hypothesis that the single strand is participating in a binding tertiary interaction indicative of a quadruplex.

The mass accuracy data obtained for this sample provides support for the hypothesis that the second peak present in the chromatograms for the RP-HPLC methods described in Example 6 for the separation of the antisense strand samples is in fact G-quadruplex. It is interesting to note that in the single strand sample, the UV peak corresponding to this higher order structure does not produce any MS signal in the Total Ion Chromatogram (TIC). A sample enriched for the G-quadruplex was required to observe MS signal corresponding to the G-quadruplex.

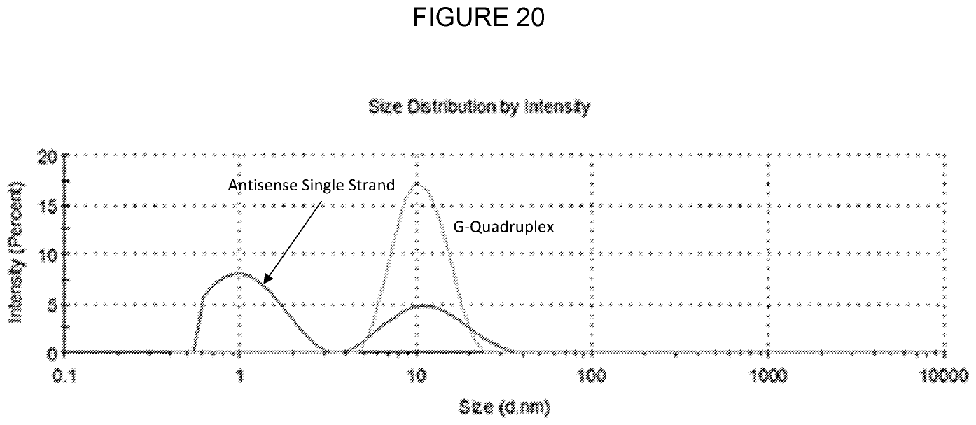

Dynamic Light Scattering (DLS): DLS analysis was performed to investigate the particle size distribution present in both the antisense single strand and G-quadruplex samples. Analysis of the antisense single strand sample indicated two particle size distributions, >2 nm and 11-12 nm (FIG. 20). In contrast, the proposed G-quadruplex sample, prepared with cation to preferentially select for a higher order structure, only contained a single particle size distribution of ˜11 nm. The presence of some larger sized particles in the single antisense strand sample is consistent with the observation of a low level second peak observed during analysis of antisense strand samples using the RP-HPLC methods described in Example 6. Furthermore, a very low-level single strand peak can be observed in the proposed G-quadruplex sample, but this is clearly minimal as no smaller particles are observed by DLS indicating that the vast majority of the antisense strands are participating in a higher order structure (i.e. quadruplex).

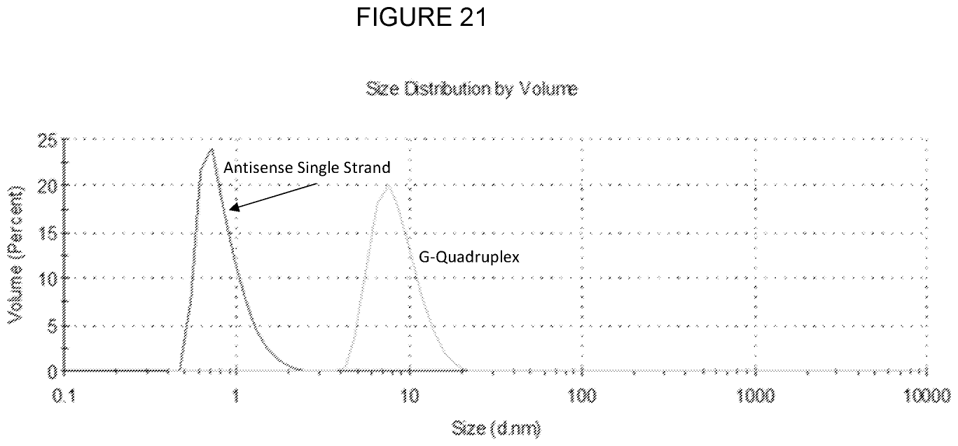

Looking at the particle size distribution by volume, it remains clear that the majority of the single strand sample is predominately sized >2 nm as can be seen in FIG. 21.

SEQUENCE TABLE

| SEQ ID | | | |
|---|---|---|---|
| NO: | Short Name | Sequence (5′ → 3′) | Description |
| 1 | Sense Strand | CAG CCC CUU AUU GUU AUA | |
| | Unmodified | CGA | |
| | Sequence | | |
| 2 | Antisense | UCG UAU AAC AAU AAG GGG | |
| | Strand | CUG | |
| | Unmodified | | |
| | Sequence | | |
| 3 | Sense Strand | CAG CCC CUU AUU GUU AUA | Wherein: |
| | modified | CGA | Each of the nucleotides at positions 1-8 |
| | Sequence | | and 12-20 is a 2′-O-methyl nucleotide |
| | | | Each of the nucleotides at positions 9- |
| | | | 11 is a 2′-deoxy-2′-fluoro nucleotide |
| | | | the nucleotide at position 21 is a 3′-3′ |
| | | | linked deoxynucleotide |
| | | | the nucleotides at positions 1 and 2 are |
| | | | linked by a phosphorothioate bond |
| | | | the nucleotides at positions 20 and 21 |
| | | | are linked by a phosphorothioate bond |
| | | | The nucleotide at position 1 is attached |
| | | | to R1 by a phosphorothioate bond, |
| | | | wherein R1 is an n-acetylgalactosamine |
| | | | glycopeptide |
| | | | The nucleotide at position 21 is an |
| | | | inverted deoxyadenosine (3′-3′ linked |
| | | | nucleotide) |
| 4 | Antisense | UCG UAU AAC AAU AAG GGG | Wherein: |
| | Strand | CUG | Each of the nucleotides at positions 1, |
| | modified | | 3, 5, 7-11, 13, 15, 17, 19, and 21 is a 2′- |
| | Sequence | | O-methyl nucleotide |
| | | | Each of the nucleotides at positions 2, |
| | | | 4, 6, 12, 14, 16, 18, and 20 is a 2′- |
| | | | deoxy-2′-fluoro nucleotide |
| | | | the nucleotides at positions 1 and 2 are |
| | | | linked by a phosphorothioate bond |
| | | | the nucleotides at positions 2 and 3 are |
| | | | linked by a phosphorothioate bond |
| | | | the nucleotides at positions 19 and 20 |
| | | | are linked by a phosphorothioate bond |
| | | | the nucleotides at positions |
| | | | 20 and 21 are linked |
| | | | by a phosphorothioate bond |
| 5 | | TTAGGG | |
| 6 | | GGGGCC | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of separating molecular species of a guanine-rich oligonucleotide from a mixture of molecular species, wherein at least one molecular species of the mixture is a quadruplex formed from the guanine-rich oligonucleotide, said method comprising:
a. applying the mixture to a chromatographic matrix comprising a hydrophobic ligand, wherein said hydrophobic ligand comprises C4 to C8 alkyl chains, wherein molecular species bind to the hydrophobic ligand;
b. applying a mobile phase which comprises a gradient of acetate and a gradient of acetonitrile to the chromatographic matrix to elute molecular species of the guanine-rich oligonucleotide, wherein the guanine-rich oligonucleotide elutes in a first set of elution fractions and the quadruplex elutes in a second set of elution fractions.

2. The method of claim 1, wherein the guanine-rich oligonucleotide is a sense strand or an antisense strand of a small interfering RNA (siRNA).

3. The method of claim 1 or 2, wherein the mixture comprises a single-stranded molecular species and/or a double-stranded molecular species.

4. The method of claim 3, wherein the mixture comprises one or more molecular species selected from the group consisting of: an antisense single strand, a sense single strand, a duplex, and a quadruplex.

5. The method of claim 4, wherein the guanine-rich oligonucleotide is the antisense single strand.

6. The method of claim 4 or 5, wherein the duplex comprises the antisense single strand and the sense single strand.

7. The method of any one of claims 4 to 6, wherein the mixture comprises the following molecular species: an antisense single strand, a sense single strand, a duplex, and a quadruplex.

8. The method of any one of the preceding claims, wherein each molecular species elutes in a fraction separate from that of another molecular species.

9. The method of claim 8, wherein the mixture comprises an antisense single strand, a sense single strand, a duplex, and a quadruplex and the duplex elutes in a first set of elution fractions, the sense strand elutes in a second set of elution fractions, the antisense strand elutes in third set of elution fractions, and the quadruplex elutes in a fourth set of elution fractions.

10. The method of any one of the preceding claims, wherein the LOQ of each molecular species is about 0.03 mg/mL to about 0.08 mg/mL.

11. The method of any one of claims 8 to 10, wherein the resolution of the separation of the peaks of each molecular species is at least or about 1.0, optionally, at least or about 1.2.

12. The method of claim 11, wherein the resolution of the separation of the peaks of each molecular species is at least or about 2.0, optionally, at least or about 2.4.

13. The method of any one of the preceding claims, wherein the mixture is prepared in a solution comprising one or more of: water, a source of acetate, a source of potassium, and sodium chloride.

14. The method of claim 13, wherein the source of acetate is ammonium acetate, sodium acetate, or potassium acetate.

15. The method of claim 13, wherein the source of potassium is potassium phosphate.

16. The method of any one of claims 13 to 15, wherein the solution comprises about 50 mM to about 150 mM acetate or potassium.

17. The method of claim 16, wherein the solution comprises about 75 mM to about 100 mM of ammonium acetate, sodium acetate, or potassium acetate.

18. The method of any one of claims 13 to 17, wherein the solution comprises potassium phosphate and sodium chloride.

19. The method of claim 1, wherein the mixture is prepared in water, optionally, purified, deionized water.

20. The method of any one of the preceding claims, wherein the hydrophobic ligand comprises C4 alkyl chains, C6 alkyl chains, or C8 alkyl chains.

21. The method of claim 20, wherein the hydrophobic ligand comprises C4 alkyl chains.

22. The method of any one of the preceding claims, wherein the chromatographic matrix is housed in a chromatographic column having an internal diameter of 2.1 mm and/or a column length of about 50 mm.

23. The method of any one of the preceding claims, wherein the column temperature is about 20° C. to about 35° C.

24. The method of claim 23, wherein the column temperature is about 29° C. to about 31° C., optionally, about 30° C.

25. The method of any one of the preceding claims, wherein the matrix comprises 1.7 ethylene bridged hybrid (BEH) particles.

26. The method of any one of the preceding claims, wherein the gradient of acetate is made with an acetate stock solution comprising about 50 mM to about 150 mM acetate.

27. The method of claim 26, wherein the acetate stock solution comprises about 70 mM to about 80 mM acetate, optionally, about 75 mM acetate.

28. The method of claim 26, wherein the acetate stock solution comprises about 90 mM to about 110 mM acetate, optionally, about 100 mM acetate.

29. The method of any one of the preceding claims, wherein the acetate is ammonium acetate, sodium acetate or potassium acetate.

30. The method of any one of claims 26 to 29, wherein the pH of the acetate stock solution is about 6.5 to about 7.0.

31. The method of claim 30, wherein the pH of the acetate stock solution is between 5.0 to 8.5, about 6.6, about 6.7, about 6.8, about 6.9 or about 7.0.

32. The method of any one of claims 27 to 31, wherein the acetate stock solution is 75 mM ammonium acetate in water having a pH of 6.7±0.1.

33. The method of any one of the preceding claims, wherein the gradient of acetonitrile is made with an acetonitrile stock solution and the acetonitrile stock solution is 100% acetonitrile.

34. The method of any one of the preceding claims, wherein the mobile phase comprises a decreasing concentration gradient of the acetate and an increasing concentration gradient of acetonitrile.

35. The method of any one of the preceding claims, wherein the gradient of acetate starts with a maximum concentration and gradually decreases to a minimum concentration over a first time period.

36. The method of claim 35, wherein the first time period is about 18 to about 19 minutes.

37. The method of claim 35, wherein the first time period is about 22 to about 26 minutes.

38. The method of any one of claims 35-37, wherein after the first time period, the acetate concentration in the mobile phase increases to the maximum concentration of acetate.

39. The method of claim 38, wherein the acetate increases to the maximum concentration of acetate about 0.1 to about 3 minutes after the gradient reaches the minimum concentration of acetate.

40. The method of any one of the preceding claims, wherein the gradient of acetonitrile starts with a minimum concentration and gradually increases to a maximum concentration over the first time period.

41. The method of claim 40, wherein after the first time period, the acetonitrile concentration in the mobile phase decreases to the minimum concentration of acetonitrile.

42. The method of claim 41, wherein the concentration of acetonitrile decreases to the minimum concentration about 0.1 to about 3 minutes after the gradient of acetonitrile reaches the maximum concentration of acetonitrile.

43. The method of any one of claims 1 to 42, comprising applying the mobile phase to the chromatographic matrix according to the following conditions:

| Time (min) | Acetate (%) | Acetonitrile (%) |
|---|---|---|
| 0 | 93 | 7 |
| 5 | 88 | 12 |
| 8 | 88 | 12 |
| 11 | 86 | 14 |
| 18 | 70 | 30 |
| 19 | 70 | 30 |
| 21 | 93 | 7 |
| 26 | 93 | 7 |

44. The method of any one of claims 1 to 42, comprising applying the mobile phase to the chromatographic matrix according to the following conditions:

| Time (min) | Acetate (%) | Acetonitrile (%) |
|---|---|---|
| 0 | 92 | 8 |
| 2 | 90 | 10 |
| 18 | 86 | 14 |
| 26 | 70 | 30 |
| 26.1 | 92 | 8 |
| 30 | 92 | 8 |

45. The method of any one of claims 1 to 42, comprising applying the mobile phase to the chromatographic matrix according to the following conditions:

| Time (min) | Acetate %) | Acetonitrile (%) |
|---|---|---|
| 0.0 | 92 | 8 |
| 2.0 | 90 | 10 |
| 18.0 | 86 | 14 |
| 22.0 | 78 | 22 |
| 22.1 | 20 | 80 |
| 24.0 | 20 | 80 |
| 24.1 | 92 | 8 |
| 30.0 | 92 | 8 |

46. The method of any one of the preceding claims, wherein the mobile phase does not comprise a cationic ion pairing agent.

47. The method of any one of the preceding claims, wherein the total run time is at least about 25 minutes and less than 40 minutes.

48. The method of any one of the preceding claims, wherein the total run time is less than 35 minutes, optionally, less than or equal to 30 minutes.

49. The method of claim 48, wherein the run time is about 26 minutes.

50. The method of any one of the preceding claims, wherein the flow rate of the mobile phase is about 0.5 ml/min to about 1 ml/min.

51. The method of any one of the preceding claims, wherein the flow rate of the mobile phase is about 0.7 ml/min to about 0.8 ml/min.

52. The method of any one of the preceding claims, comprising monitoring elution of molecular species using an ultraviolet detector.

53. The method of any one of the preceding claims, which is a non-denaturing method.

54. The method of any one of the preceding claims, further comprising collecting the elution fractions into separate containers over a time period.

55. The method of any one of the preceding claims, wherein the guanine-rich oligonucleotide comprises about 19 to about 23 nucleotides.

56. The method of any one of the preceding claims, wherein the guanine-rich oligonucleotide and one or more of the molecular species thereof in the mixture comprises one or more modified nucleotides.

57. The method of claim 56, wherein the one or more modified nucleotides are 2′-modified nucleotides.

58. The method of claim 57, wherein the 2′-modified nucleotides are 2′-O-methyl modified nucleotides, 2′-fluoro modified nucleotides, deoxynucleotides, or combinations thereof.

59. The method of any one of the preceding claims, wherein the guanine-rich oligonucleotide and one or more of the molecular species thereof in the mixture comprises synthetic intemucleotide linkages.

60. The method of claim 59, wherein the synthetic intemucleotide linkage is a phosphorothioate linkage.

61. The method of any one of the preceding claims, wherein the guanine-rich oligonucleotide comprises the sequence of SEQ ID NO: 2.

62. The method of any one of the preceding claims, wherein the guanine-rich oligonucleotide comprises the sequence of modified nucleotides according to SEQ ID NO: 4.

63. A method of separating molecular species of a guanine-rich oligonucleotide from a mixture of molecular species, wherein the molecular species of the mixture are a quadruplex formed from the guanine-rich oligonucleotide, the guanine-rich oligonucleotide, a duplex comprising the guanine-rich oligonucleotide and the complement strand thereof, and the complement strand, said method comprising:
a. applying the mixture to a chromatographic matrix comprising a hydrophobic ligand, wherein said hydrophobic ligand comprises C4 to C8 alkyl chains, wherein molecular species bind to the hydrophobic ligand;
b. applying a mobile phase which comprises a decreasing concentration gradient of acetate and an increasing concentration gradient of acetonitrile to the chromatographic matrix to elute molecular species of the guanine-rich oligonucleotide, wherein each of the quadruplex, the guanine-rich oligonucleotide, the duplex and the complement strand separately elute from the chromatographic matrix.

64. The method of claim 63, comprising applying the mobile phase to the chromatographic matrix according to the following conditions:

| Time (min) | Acetate (%) | Acetonitrile (%) |
|---|---|---|
| 0.0 | 92 | 8 |
| 2.0 | 90 | 10 |
| 18.0 | 86 | 14 |
| 22.0 | 78 | 22 |
| 22.1 | 20 | 80 |
| 24.0 | 20 | 80 |
| 24.1 | 92 | 8 |
| 30.0 | 92 | 8 |

65. The method of claim 63 or 64, wherein the resolution of the separation of the peaks of each molecular species is at least or about 2.0, optionally, at least or about 2.4.

66. The method of claim 65, wherein the resolution of the separation of the peaks of each molecular species is at least or about 3.0 or at least or about 4.0.

67. The method of any one of claims 63-66, wherein the LOQ of each molecular species is about 0.03 mg/mL to about 0.08 mg/mL.

68. A method of determining the purity of a sample comprising a guanine-rich oligonucleotide drug substance or drug product, comprising separating molecular species of the guanine-rich oligonucleotide in accordance with any one of claims 1-67.

69. The method of claim 68, wherein the sample is an in-process sample.

70. The method of claim 68, wherein the sample is a lot sample.

71. A method of testing stability of a guanine-rich oligonucleotide drug substance or drug product, comprising applying stress to a sample comprising the guanine-rich oligonucleotide drug substance or drug product and determining the purity of the sample according to claim 68.


SEQUENCE LISTING (4 sequences)

SEQ ID NO: 1 - RNA, 21 nt, synthetic construct
  misc_feature 1..21: note = Synthetic polynucleotide
  source 1..21: mol_type = other RNA
cagccccttattgttatacga

SEQ ID NO: 2 - RNA, 21 nt, synthetic construct
  misc_feature 1..21: note = Synthetic polynucleotide
  source 1..21: mol_type = other RNA
tcgtataacaataaggggctg

SEQ ID NO: 3 - RNA, 21 nt, synthetic construct
  misc_feature 1..21: note = Synthetic modified polynucleotide
  modified_base 1..21: mod_base = OTHER; note = combination of 2'-fluoro, 2'-O-methyl, inverted deoxyadenosine,phosphorothioate linked ribonucleotides
  source 1..21: mol_type = other RNA
cagccccttattgttatacga

SEQ ID NO: 4 - RNA, 21 nt, synthetic construct
  misc_feature 1..21: note = Synthetic modified polynucleotide
  modified_base 1..21: mod_base = OTHER; note = combination of 2'-fluoro, 2'-O-methyl, phosphorothioate linked ribonucleotides
  source 1..21: mol_type = other RNA
tcgtataacaataaggggctg